(19) United States
(12) Patent Application Publication
(10) Pub. No.: US 2026/0183434 A1
(43) Pub. Date: Jul. 2, 2026

(54) NEW SENESCENCE TRACER GENERATION

(71) Applicant: Eberhard Karls Universitaet Tuebingen Medizinische Fakultaet, Tuebingen (DE)

(72) Inventors: Jonathan Cotton, Kusterdingen (DE); Bernd Pichler, Fernhag (DE); Paul Johannes Oskar Maria Kinzler, Horb am Neckar (DE); Adriana Di Nanni, Tuebingen (DE); Francisco José Reche Pérez, Kirchheim unter Teck (DE); Marcel André Krueger, Reutlingen (DE); Benyuan Zhou, Tuebingen (DE)

(21) Appl. No.: 19/323,898

(22) Filed: Sep. 9, 2025

Related U.S. Application Data
(63) Continuation of application No. PCT/EP2024/056676, filed on Mar. 13, 2024.

(30) Foreign Application Priority Data
Mar. 13, 2023 (DE) 10 2023 106 170.4

(51) Int. Cl. A61K 51/04; C07H 15/22; G01N 33/50

(52) U.S. Cl. CPC A61K 51/0491; C07H 15/22; G01N 33/5023; A61K 2123/00

(57) ABSTRACT

The present invention relates to compounds useful for visualizing cell senescence in vitro and in vivo, the preparation of said compounds and their use. In particular, the present invention pertains to novel glycosides for use as senescence tracers in vitro and in vivo.

### CROSS REFERENCES TO RELATED APPLICATIONS

This application is a continuation of international patent application PCT/EP2024/056676, filed on Mar. 13, 2024 designating the U.S., which international patent application was published in the German language and claims priority to German patent application DE 10 2023 106 170.4, filed on Mar. 13, 2023. The entire contents of these priority applications are incorporated herein by reference.

### BACKGROUND

The present invention relates to compounds useful for visualizing cell senescence in vitro and in vivo, the preparation of said compounds and their use. In particular, the present invention pertains to novel glycosides for use as senescence tracers in vitro and in vivo.

Cell senescence is the biological process by which cells exit the growth cycle. Cell senescence was first characterized in fibroblasts, which could only be subjected to a limited number of passages, before growth became permanently arrested. This phenomenon is named the Hayflick limit and serves to explain the physiological course of aging. It is accompanied by distinct change in metabolic pathways (Roninson, E. B., Tumor Cell Senescence in Cancer Treatment, Cancer Res., 2003, 63:2705-2715).

Senescent cells exhibit a senescence associated secretory phenotype, containing pro-inflammatory cytokines and growth factors. In certain instances, the removal of senescent tissues can convey great benefits to a patient. Senescence is recognized to play an important role in cancer treatment and therapy resistance. Treatment-associated senescence marks as stable clinical end point, and thus can be a measurement of chemotherapeutic success. The detection of senescence cells might also offer diagnostic opportunities for detecting pre-neoplastic lesions (Roninson, E. B., Tumor Cell Senescence in Cancer Treatment, Cancer Res., 2003, 63:2705-2715; and Campisi, J. and d'Adda di Fagagna, F., Cellular Senescence: when bad things happen to good cells, Nature Reviews Molecular Cell Biology, 2007, 8:729-740).

The most widely used surrogate marker for senescence cells is the senescence-associated beta-galactosidase (Roninson, E. B., Tumor Cell Senescence in Cancer Treatment, Cancer Res., 2003, 63:2705-2715). Such beta-galactosidase substrates have, however, the drawback that they merely may be used ex vivo and in vitro for following beta-galactosidase expression.

In recent years beta-galactosidase specific PET (Positron Emission Tomography) tracer for in vivo imaging of tumor senescence have been presented. For instance, the PET tracer [¹⁸F]FPyGal can enable the non-invasive imaging of ß-galactosidase as a surrogate marker for senescence. Drawbacks of said compound reside in its limited retention time.

The present invention seeks to overcome the problems associated with prior art compounds.

There exists the need for further compounds that can act as tracers for cell senescence which may be used in vitro and in vivo at the same time. There is also the need for radioactive tracers for cell senescence with an elongated retention time in cells. Further, there is a need for radioactive tracers for cell senescence that accumulate in senescent cells at localized portions thereof. There is also a need in developing improved methods in the treatment of disorders related to cell senescence, such as cancer, employing a lower radioactive dose and/or employing the same dose with higher resolution/improved imaging. A further objective of the present invention resides in alternative methods for selectively eliminating senescent cells in vivo. Still a further objective of the present invention is the provision of alternative means for determining the efficiency of cancer treatment.

### SUMMARY

The present invention is based on the finding that some radioactive tracers can be retained inside cells taking use of different mechanisms, in particular a self-immolation mechanism that may involve chemical binding to cellular constituents or sorption processes. Thereby, the retention time of hexose derivatives carrying radioactive labels in senescent cells can be actively prolonged, thereby enabling or facilitating the labelling and detection of said senescent cells both in vitro and in vivo.

Therefore, the present invention provides a compound of the formula:

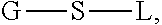

or a stereoisomer, enantiomer, tautomer, prodrug and/or a pharmaceutically acceptable salt thereof,
wherein
G is a glycoside, and a substituted or unsubstituted C₁-C₅ alkyl derivative thereof and/or a substituted or unsubstituted N-acetyl derivative thereof having a glycosidic bond to S, wherein * represents the binding site between G and S,
S is selected from the group consisting of

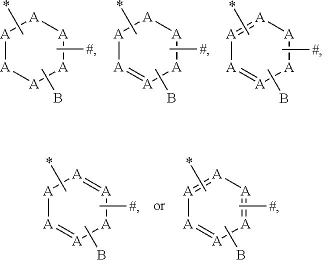

A is independently selected from the group consisting of C, S, N, and O, with the proviso that at least 3 C-atoms are present;
#represents the binding site between S and L;
L is selected from the group consisting of

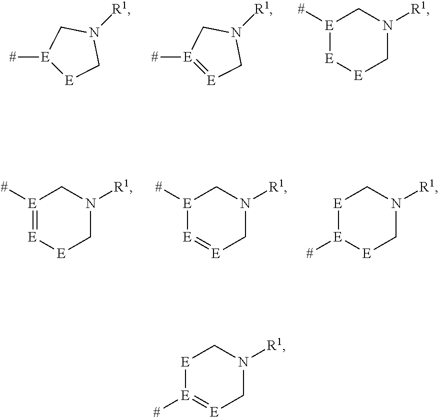

#—CH₂OR², or #—CH₂OOCNHR²;
E is independently selected from the group consisting of N, NH, C, CH, and CH₂;
B and R¹ are independently selected from the group consisting of R³Z, H, F, Cl, Br, I, NO₂, OR⁴, NR⁴R⁵, and substituted or unsubstituted C₁-C₅ alkyl, provided that at least one R³Z is present;
R² is selected from the group consisting of substituted or unsubstituted alkyl, substituted or unsubstituted alkenyl, substituted or unsubstituted aryl, and substituted or unsubstituted heteroaryl; and
R³Z is selected from the group consisting of (CH₂)ₘZ, [(CH₂)ₘO(CH₂)ₙ]ₒZ, [(CH₂)ₘCO(CH₂)ₙ]ₒZ, [(CH₂)ₘOOC(CH₂)ₙ]ₒZ, [(CH₂)ₘNR⁴(CH₂)ₙ]ₒZ, wherein m is 0 to 5, and n and o are independently from each other integers from 1 to 5;
R⁴ and R⁵ are independently selected from the group consisting of H or substituted or unsubstituted C₁-C₅ alkyl; and
Z is a radioactive detectable label, a radioactive therapeutic residue, a chelator coordinating a radioactive detectable label or a chelator coordinating a radioactive therapeutic residue.

It has been surprisingly found that the present compounds are highly and selectively enriched in senescent cells. Without wishing to be bound by theory, it is assumed that based on the attachment mechanism of the present compounds, said compounds are accumulated in localized cellular portions, e.g., organelles, of the senescent cells. This localized enrichment within cells facilitates in vivo targeting and removal of cells, particularly tumors. In general, the use of the present radioactive labels confers the advantage of highly enhanced sensitivity versus conventional labeling strategies. This is in part due to the high tissue penetration of the resulting gamma photons and very high sensitivity of the radio detectors used. The present compounds thereby offer the possibility to quantitatively, selectively and sensitively detect senescent cells, particularly tumors, in vivo. This highly selective enrichment in senescent cells may be used in diagnosis or treatment of senescence related disorders by employing radioactive detectable labels or radioactive therapeutic residues in the present compounds.

Another advantage resides in the possibility to treat tumors with non-invasive techniques, e.g., by administering the present compounds exhibiting a radioactive therapeutic residue, which are almost exclusively incorporated in tumor tissue. A further advantage of the use of the present compounds resides in their flexible use. For instance, the present compounds may be easily adapted to the changing requirements in the clinical field since the present compound may be easily provided with another radioactive detectable label or radioactive therapeutic residue.

Further advantages are that radiolabeling can take place at the final synthesis steps and can also be carried out in a relatively simple manner. This allows the final synthesis steps to be carried out at or near the site of administration of the present compound, such as in a hospital. This makes it possible to administer compounds with a relatively short half-life, such as ¹⁸F or ⁶⁴Cu. Furthermore, the facility in which the compounds are to be administered does not require a fully equipped laboratory for the initial synthesis of the compounds of the invention.

The compounds of the invention may, depending on their structure, exist in stereoisomeric forms (enantiomers, diastereomers). The invention therefore also encompasses the enantiomers or diastereomers and respective mixtures thereof. The stereoisomerically uniform constituents can be isolated in a known manner from such mixtures of enantiomers and/or diastereomers. If the compounds of the present invention may occur in tautomeric forms, the present invention encompasses all tautomeric forms. Tautomers are structural or constitutional isomers of the present compounds that readily interconvert. The present compound may be also in form of a prodrug. A prodrug is a pharmacologically inactive compound of the present invention that, after intake, is metabolized into a pharmacologically active drug.

The salts preferred for the purposes of the present invention are pharmaceutically acceptable salts of the compounds of the invention. However, also encompassed are salts which are themselves not suitable for pharmaceutical applications but can be used for example for the isolation or purification of the compounds of the invention.

Salts in general are composed of related numbers of cations and anions so that the product is electrically neutral. It will be appreciated that a respective counterion is subject to the preparation process of the salt and needs not to be expressively mentioned.

Pharmaceutically acceptable salts as well as the preparation thereof are well-known in the art. Types and preparation of such pharmaceutically acceptable salts may be derived from Stahl, P. H. and Wermuth, C. G., Handbook of Pharmaceutical Salts Properties, Selection and Use, Weinheim/Zurich: Wiley-VCH, 2011; and Gupta D., et al., Salts of Therapeutic Agents: Chemical, Physicochemical, and Biological Considerations, Molecules. 2018 July; 23(7): 1719, the contents of which are included by way of reference in their entirety.

Examples of pharmaceutically acceptable salts of the present compounds include salts of inorganic basis like ammonium salts, alkali metal salts, alkaline earth metal salts, salts of organic basis, or salts with basic amino acids. Also included are inorganic acids or salts with acidic amino acids.

It will be appreciated that the compounds of the present invention as well as the salts thereof may be present in form of solvates. In such a case, the present compounds or salts, particularly the pharmaceutically acceptable salts, thereof form in the solid or liquid state a complex by coordination with solvent molecules. Hydrates are a specific form of solvates in which the coordination displays with water.

A “glycoside” generally refers to is a molecule in which a sugar is bound to another residue or group via a glycosidic bond or linkage. The glycosides encompass hexoses, including aldohexoses, such as allose, altrose, glucose, mannose gulose, idose, galactose, and talose, and ketohexoses, such as psicose, fructose, sorbose and tagatose. The glycosides are preferably D-isomers. An example of a particularly preferred glycoside is β-D-galactosidase.

The glycoside can be present as such, as a substituted or unsubstituted C₁-C₅ alkyl derivative thereof and/or as a substituted or unsubstituted N-acetyl derivative thereof. Hence, one or more hydroxyl groups can be unmodified, modified by a substituted or unsubstituted C₁-C₅ alkyl residue as defined hereinafter or substituted with a substituted or unsubstituted N-acetyl residue. It will be appreciated that this will give raise to a plurality of different glycoside derivatives, including, e.g., a substituted or unsubstituted C₁-C₅ alkyl derivative having a single substituted or unsubstituted C₁-C₅ alkyl residue, e.g., at the last hydroxyl group, a substituted or unsubstituted N-acetyl derivative having a single substituted or unsubstituted N-acetyl substitution, e.g., in vicinity to the glycosidic bond. However, glycoside derivatives having one or more substituted or unsubstituted C₁-C₅ alkyl residues and/or substituted or unsubstituted N-acetyl residues are encompassed as well.

In case the glycoside is a substituted or unsubstituted C₁-C₅ alkyl derivative thereof and/or a substituted or unsubstituted N-acetyl derivative thereof, prolonged retention time of the present compound in comparison to the unmodified glycoside may be expected due to an impeded enzymatic cleavage. Hence, said compounds may be present in higher levels within senescent cells.

The term “alkyl” generally refers to branched or straight-chain alkyl, preferably C₁-C₁₈ alkyl, such as C₁-C₁₆ alkyl, C₁-C₁₄ alkyl, C₁-C₁₂ alkyl, C₁-C₁₀ alkyl, C₁-C₈ alkyl, C₁-C₆ alkyl, more preferably C₁-C₅ alkyl. Examples of C₁-C₅ alkyl include methyl, ethyl, propyl, butyl, isopropyl, isobutyl, tert. butyl, n-pentyl, 2-pentyl, 3-pentyl, 2-methylbutyl, 3-methylbutyl, 3-methylbut-2-yl, 2-methylbut-2-yl, and 2,2-dimethylpropyl.

The term “alkenyl” generally refers to branched or straight-chain alkyl residues that are partially or complete saturated. Hence, an alkenyl residue has at least one C—C double or triple bond.

The term “aryl” means an essentially aromatic monocyclic or polycyclic ring comprising carbon and hydrogen atoms. Preferably, the aromatic ring may fulfill the Hückel's rule. Preferably, the aryl group is a monocyclic or bicyclic ring, more preferably, a monocyclic ring, wherein the ring comprises from 4 to 8 carbon atoms. More preferably the molecular weight of an aryl residue has a molecular weight of ≤500 g/mol, such as ≤400 g/mol, ≤300 g/mol or ≤200 g/mol.

The term “heteroaryl” means an essentially aromatic monocyclic or polycyclic ring comprising carbon and hydrogen atoms and at least one heteroatom, preferably, 1 to 4 heteroatoms, such as 3 heteroatoms, 2 heteroatoms, ore one heteroatom, independently selected from nitrogen, oxygen, and sulfur. Preferably, then heteroaryl may fulfill the Hückel's rule. Preferably, the heteroaryl group is a monocyclic or bicyclic ring, more preferably, a monocyclic ring, wherein the ring comprises from 2 to 6 carbon atoms and from 1 to 3 heteroatoms. Preferably the molecular weight of a heteroaryl residue has a molecular weight of ≤500 g/mol, such as ≤400 g/mol, ≤300 g/mol or ≤200 g/mol.

The term “substituted” as used herein refers to one or more residues connected to alkyl, such as C₁-C₅ alkyl, cycloalkyl and heterocycloalkyl and/or a modification to the carbon chain by introducing one or more heteroatoms, such as N; S or O. A residue may be furthermore selected from the group consisting of fluorine, chlorine, bromine, and iodine, methyl, ethyl or functional groups, such as a hydroxyl group, an amino group, an O-alkyl group, particularly C₁-C₅ O-alkyl, nitro, or carboxyl group. The overall molecular weight of residues connected to particular alkyl, such as C₁—C alkyl, cycloalkyl or heterocycloalkyl moieties is preferably ≤250 g/mol, more preferably ≤150 g/mol or ≤100 g/mol. Examples of preferred substituted to C₁-C₅ alkyl residues encompass compounds such as CH₂—O—CH₂, C₂H₄—O—CH₂, CH₂—O—CH₂—O—CH₂, CH₂—O—CH₂—O—CH₂, C₂H₄—O—CH₂—O—CH₂, CH₂—O— C₂H₄—O—CH₂, C₂H₄—O—C₂H₄—O—CH₂, C₂H₄—O—CH₂—O— C₂H₄, CH₂—O—CH₂—O—CH₂—O—CH₂, C₂H₄—O—CH₂—O—CH₂—O—CH₂, and CH₂—O— C₂H₄—O—CH₂—O—CH₂

The term “halogen” or halo refers to fluorine, chlorine, bromine or iodine; preferably fluorine, chlorine or bromine. In some embodiments the term halogen or halo preferably refers to ⁷⁶Br, ⁷⁵Br, ¹⁹F and ¹⁸F.

The term “methyl halogen” refers to a methyl group having a single fluorine, chlorine, bromine or iodine, or two or three halogens which are independently selected from fluorine, chlorine, bromine or iodine. In some embodiments the methyl halogen exhibits preferably ⁷⁶Br, ⁷⁵Br, ¹⁹F and ¹⁸F.

The term “radioactive detectable label” encompasses for instance ¹¹C, ⁴⁰K, ¹³N, ¹⁵O, ¹⁸F, ⁷⁵Br, ⁷⁶Br, ⁸²Rb, ⁶⁸Ga, ⁶⁴Cu, ⁶²Cu, ⁸⁹Zr, ¹²³I, ¹²⁴I, ¹²⁵I, ¹³¹I, ²¹⁰At, ²¹¹At and ¹¹¹In. Preferred radioactive detectable labels are ¹¹C, ¹⁸F, ⁶⁸Ga, ⁶⁴Cu, and ¹²⁴I. The radioactive detectable label allows quantitatively and/or qualitatively assessment of senescent cells as well as their location particularly in vivo. Senescent tissue may be properly identified and for instance surgically removed with high selectivity. ¹⁸F is particularly preferred because its decay results in a non-radioactive element, decomposition of the present compound present in vivo, and removal from the cell without further damage to the cell or the organism to which the cell belongs.

Alternatively, a complex containing such an atom in a coordinated form is encompassed. In such a case the complex may be also denominated a chelator coordinating a radioactive detectable label. A (coordination) complex consists of a central atom or ion, which is usually metallic, and a surrounding array of ligands or complexing agents. Usually, the central atom or ion is the radioactive detectable label. Residue Z therefore encompasses the central atom or ion as well as the surrounding array of ligands or complexing agents. Examples of such chelators coordinating a radioactive detectable label are well known to the skilled person. Preferably such a chelator is an amino acid, preferably a proteinogenic/natural amino acid, such as histidine. Other suitable examples of chelators comprise DOTA, NOTA, NODAGA and desferrioxamine, such as desferrioxamine B.

The term “radioactive therapeutic residue” as used herein refers to an atom, such as ³²P, ⁶⁰Co, ⁸⁹Sr, ¹⁸⁶Re and ¹⁵³Sm. Other examples of radioactive therapeutic residues encompass ¹²⁵I and ¹³¹I, which have been already mentioned as examples for radioactive detectable labels, as well as ⁸⁶Y, ¹¹¹In, ¹⁷⁷Lu and ⁶⁷Cu. Alternatively, a complex containing such an atom in a coordinated form is encompassed. In such a case the complex may be also denominated a chelator coordinating a radioactive therapeutic residue. The use of the present compounds with therapeutic residues in treatment of disorders associated with cell senescence, particularly cancer, combines the advantage of target selectivity with that of being systemic, as with chemotherapy, and it may be used as part of a therapeutic strategy with curative intent or for disease control and palliation.

Particularly, beta-galactosidases of human or animal origin have already proven to tolerate large residues attached to the glycoside without affecting the enzyme activity. Hence, there is a reasonable expectation that present compounds having a bulky radioactive therapeutic residue are convertible by the enzyme.

It will be appreciated that respective radioactive therapeutic residues may be generated and attached to the present compounds in the same manner as radioactive detectable labels. Preferably, therapeutic residue and detectable label are identical permitting diagnosis and treatment of disorders associated with cell senescence, particularly cancer.

In formulae, the group which is represented by G, the end point of the line adjacent to which there is a *, is not a carbon atom or a CH₂ group but rather a component of the bond to the atom to which G is attached.

In formulae, the group which is represented by S, the end point of the line adjacent to which there is a * or #, is not a carbon atom or a CH₂ group but rather a component of the bond to the atom to which S is attached.

In formulae, the group which is represented by L, the end point of the line adjacent to which there is a #, is not a carbon atom or a CH₂ group but rather a component of the bond to the atom to which L is attached.

In the context of the present invention, the expression “have/contain” or “having/containing” designates an open enumeration and does not exclude other components apart from the expressly named components.

In the context of the present invention, the expression “consists of” or “consisting of” designates a closed enumeration and excludes any other components apart from the expressly named components.

In the context of the present invention, the expression “essentially consists of” or “essentially consisting of” designates a partially closed enumeration and designates preparations which apart from the named components only have such further components as do not materially alter the character of the preparation according to the invention.

When in the context of the present invention a preparation is described with the use of the expression “have” or “having”, this expressly includes preparations which consist of said components or essentially consist of said components.

Radiology as used herein refers to any kind of apparatus or device, capable of producing a visual signal or an image upon detecting the present residue Z, i.e. a radioactive detectable label, a radioactive therapeutic residue, a chelator coordinating a radioactive detectable label or a chelator coordinating a radioactive therapeutic residue. Preferably the apparatus permits localization of residue Z within a mammal. Non limiting examples of radiology include positron-emissions-tomography (PET), which produces a three-dimensional image or map of functional processes in the body. The system detects pairs of gamma rays emitted indirectly by a positron-emitting radioisotope, which is introduced into the body on a metabolically active molecule, i.e. a substrate to beta-galactosidase. Images of metabolic activity in space are then reconstructed by computer analysis, which may be supported by a CT X-ray scan performed on the patient in the same session, preferably at the same time, and in the same device in order to obtain a three dimensional image enabling localization of tissue in which the prodrug is enriched. In positron emission a proton is converted via the weak force to a neutron, a positron and a neutrino. Isotopes, which undergo this so called beta plus decay, thereby emit positrons. Suitable positron-emitting radionuclides for this purpose include ¹¹C, ⁴⁰K, ¹³N, ¹⁵O, ¹⁸F, ⁷⁵Br, ⁷⁶Br, ⁸⁹Zr, ⁸²Rb, ⁶⁸Ga, ⁶²Cu and ⁶⁴Cu, of which ¹¹C and ¹⁸F are preferred. Other useful radionuclides include ¹²³I, ¹²⁴I, ¹²⁵I, ¹³¹I, ²¹⁰At, ²¹¹At and ¹¹¹In. The present compounds may be also labeled with technetium and rhenium isotopes using known chelating complexes. Methods for the generation of radionuclides as well as radio labeling of compounds are well known to the skilled person. US 2007/0273308 and WO 2007/122488, the contents of which are incorporated by way of reference in their entirety, pertain e.g. to the production of radionuclides. Radiolabeling is for example outlined in WO 2007/148089 and WO 2007/148083, the contents of which are incorporated by way of reference in their entirety.

PET is preferably coupled with a computer tomography (CT). Such PET/CT devices enable quantitative detection and allocation of the signals detected to particular tissues, i.e. a localization of the radionuclides employed and hence of the prodrugs attached thereto. Function and operation of PET/CT as well as devices are well known to the skilled person. Other suitable techniques comprise single photon emission computed tomography (SPECT) and techniques based on nuclear magnetic resonance (NMR) wherein the quantum mechanical magnetic properties of an atom's nucleus are detected.

The radiology device employed is preferably a PET device, more preferably a PET/CT device or a PET/MR device.

### BRIEF DESCRIPTION OF THE DRAWINGS

In the Figures

FIG. 1 shows a model experiment to validate self-immolation mechanism;

FIG. 2 shows an uptake experiment in BG over-expressing cells;

FIG. 3 shows uptake experiments in senescence cell models;

FIG. 4 shows dynamic PET scans of Amp19 tumor bearing mice with [¹⁸F]TFPBGal;

FIG. 5 shows an autoradiography of cx-treated (senescent) and control Amp19 tumors after administration of [¹⁸F]PIPGal; and

FIG. 6 shows an ex vivo immunohistochemistry of cx-treated (senescent) and vehicle-treated (control) Amp19 tumors.

### PREFERRED EMBODIMENTS

According to a preferred embodiment, the substituted or unsubstituted C₁-C₅ alkyl derivative of the glycoside is a substituted or unsubstituted 5-(C₁-C₅ alkyl)glycoside; and/or the substituted or unsubstituted N-acetyl derivative of the glycoside is a substituted or unsubstituted 2-(N-acetyl)glycoside, a substituted or unsubstituted 3-(N-acetyl)glycoside, or a substituted or unsubstituted poly(N-acetyl)glycoside; and/or the glycoside is selected from the group consisting of α-D-glucofuranoside, α-D-mannofuranoside, α-D-fructofuranoside, α-D-glucopyranoside, α-D-mannopyranoside, α-D-galactopyranoside, α-D-fructopyranoside, β-D-glucofuranoside, β-D-mannofuranoside, β-D-fructofuranoside, β-D-glucopyranoside, β-D-mannopyranoside, β-D-galactopyranoside, and β-D-fructopyranoside; and/or R³Z is selected from the group consisting of Z, [(CH₂)ₘO(CH₂)ₙ]ₒZ, [(CH₂)ₘCO(CH₂)ₙ]ₒZ, NR⁴(CH₂)ₙZ, wherein R⁴ is H, methyl or ethyl, wherein m, n and o are independently from each other integers from 1 to 5; and/or S is

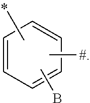

According to another preferred embodiment, wherein L is

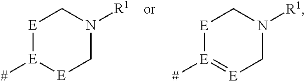

preferably

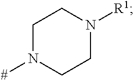

R¹ is independently selected from the group consisting of (CH₂)ₙZ, or O(CH₂)ₙZ, wherein n is 1 to 3, preferably wherein #is in meta-position or para-position to *; B is independently selected from the group consisting of H, F, Cl, Br, I, NO₂, OR⁴, NR⁴R⁵, and unsubstituted C₁-C₅ alkyl, provided that at least three H are present, wherein R⁴ and R⁵ are independently selected from the group consisting of H, methyl, or ethyl.

According to still another preferred embodiment, L is #—CH₂OR² or #—CH₂OOCNHR², preferably wherein L is in ortho-position or para-position to *; R² is substituted or unsubstituted C₁ to C₈ alkyl, substituted or unsubstituted aryl, preferably substituted naphthyl. biphenyl, or benzyl; B is independently selected from the group consisting of R³Z, H, F, Cl, Br, I, NO₂, and substituted or unsubstituted C₁-C₅ alkyl, provided that at least one R³Z at least two H are present are present; R³Z is selected from the group consisting of —Z, —[(CH₂)ₘO(CH₂)ₙ]ₒZ, —[(CH₂)ₘCO(CH₂)ₙ]ₒZ, —NH(CH₂)ₙZ, wherein m, n and o are independently from each other integers from 1 to 4, preferably wherein R³Z is in para-position to * or #; and/or R⁴ and R⁵ are independently selected from the group consisting of H and substituted or unsubstituted C₁ to C₃ alkyl.

If L is in ortho- or para-position position to *, formation of quinone methide is facilitated.

According to a preferred embodiment, the compound is

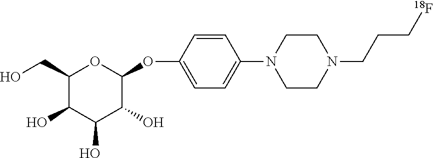 [SMILES: OC[C@H]1O[C@@H](Oc2ccc(N3CCN(CCC[18F])CC3)cc2)[C@H](O)[C@@H](O)[C@H]1O]

compound [¹⁸F]20 (also denominated (SA-)PIPGal, [¹⁸F]PIPGal, or PIPGal),

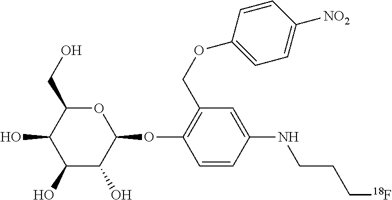 [SMILES: O=[N+]([O-])c1ccc(OCc2cc(NCCC[18F])ccc2O[C@@H]2O[C@H](CO)[C@H](O)[C@H](O)[C@H]2O)cc1]

compound [¹⁸F]9 (also denominated (SA-)NO2-ADBGal, or [¹⁸F]NO2ADBGal),

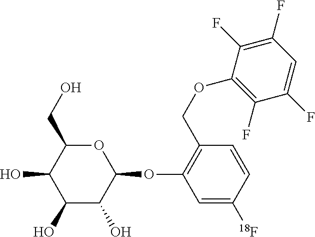 [SMILES: OC[C@H]1O[C@@H](Oc2cc([18F])ccc2COc2c(F)c(F)cc(F)c2F)[C@H](O)[C@@H](O)[C@H]1O]

compound [¹⁸F]29 (also denominated (SA-) TFPBGal, or [¹⁸F]TFPBGal), or

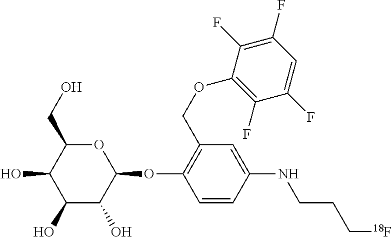 [SMILES: OC[C@H]1O[C@@H](Oc2ccc(NCCC[18F])cc2COc2c(F)c(F)cc(F)c2F)[C@H](O)[C@@H](O)[C@H]1O]

compound [¹⁸F]14 (also denominated (SA-) ADTFPBGal, or [¹⁸F]ADTFPBGal).

According to another preferred embodiment of the present invention the radioactive detectable label is selected from the group consisting of ¹¹C, ⁴⁰K, ¹³N, ¹⁵O, ¹⁸F, ⁷⁵Br, ⁷⁶Br, ⁸²Rb, ⁶⁸Ga, ⁶⁴Cu, ⁶²Cu, ⁸⁹Zr, ¹²³I, ¹²⁴I, ¹²⁵I, ¹³¹I, ²¹⁰At, ²¹¹At and ¹¹¹In, preferably ¹¹C, ¹⁸F, ⁶⁸Ga, ⁶⁴Cu, and ¹²⁴I, more preferably ¹⁸F.

According to still another preferred embodiment of the present invention the radioactive therapeutic residue is selected from the group consisting of ³²P, ⁶⁰Co, ⁶⁴Cu, ⁸⁹Sr, ⁹⁰Y, ¹⁷⁷Lu, ¹⁸⁶Re and ¹⁵³Sm.

According to a preferred embodiment of the present invention the present compound is for use in surgery. The present compound is preferably employed in combination with at least one inert, non-toxic, pharmaceutically suitable excipient.

According to a preferred embodiment of the present invention the present compound is for use as a medicament. The present compound is preferably employed in combination with at least one inert, non-toxic, pharmaceutically suitable excipient.

According to another preferred embodiment of the present invention the present compound for use in a method for detecting cell senescence.

According to another preferred embodiment of the present invention the present compound is for use in a method of determining the efficiency of cancer treatment.

The cancer to be diagnosed and/or treated by the present compounds may be selected from prostate carcinoma, colorectal carcinoma, breast cancer, lung tumors, tumors of the male or female genitourinary system, malign melanoma, cervix and throat tumor/cervical tumor, malign lymphoma, neoplasia of the hematopoietic system and musculoskeletal tumors.

According to still another preferred embodiment of the present invention the method for detecting cell senescence comprises contacting cells with the present compound.

According to still another preferred embodiment of the present invention the method for determining the efficiency of cancer treatment comprises contacting cells with the present compound.

According to still another preferred embodiment of the present invention the method is performed in vivo.

According to still another preferred embodiment of the present invention the method is performed in vitro.

The above methods can be performed both in vivo, e.g. in a human patient for monitoring the efficiency of cancer treatment, or in vitro, e.g. for screening for new medicaments.

The present compound will preferably be administered parenterally.

For this administration route, the present compounds may be administered in suitable administration forms. Parenteral administration can take place with avoiding of an absorption step (e.g. intravenous, intraarterial, intracardiac, intraspinal or intralumbar) or with inclusion of an absorption step (e.g. intramuscular, subcutaneous, intracutaneous, percutaneous or intraperitoneal). Administration forms suitable for parenteral administration are, inter alia, preparations for injection and infusion in the form of solutions, suspensions, emulsions, lyophilisates or sterile powders.

The present compounds may be converted into the stated administration forms. This can take place in a manner known per se by mixing with inert, non-toxic, pharmaceutically acceptable excipients. These excipients include inter alia carriers (for example microcrystalline cellulose, lactose, mannitol), solvents (e.g. liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g. antioxidants such as, for example, ascorbic acid), colors (e.g. inorganic pigments such as, for example, iron oxides) and taste and/or odor corrigents.

Preferably, the present compound is adapted to accumulate intracellularly. More preferably, intracellular accumulation takes place in the same cell in which the present compound is converted by the senescent associated beta-galactosidase (SABG). Intracellular accumulation may be based on a self-immolation mechanism that may involve chemical binding to cellular constituents or sorption processes. Still more preferably, intracellular accumulation includes bonding of the present compound, such as covalent bonding and/or ionic bonding. Bonding, such as covalent bonding and/or ionic bonding, to an intracellular constituent, e.g. a protein, may take place.

The principle underlying the present invention is schematically shown in schemes 1 and 2 hereinafter.

According to scheme 1, the radioactive tracer, e.g., PIPGal comprises a basic functionality, particularly a tertiary amine, in vicinity to R¹. In this regard, particularly compounds

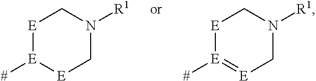

and preferably

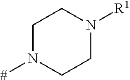

have shown good results. After senescence associated beta-galactosidase (SABG) or beta-galactosidase mediated cleavage of the glucosidase moiety, the nitrogen adjacent to residue R¹ may be easily protonated in acidic lysosomes of the target cell and trapped therein. This so-called pH trapping is presumably due to sorption, particularly adsorption, of the ammonium group to negatively charged cell components, such as negatively charged amino acids of proteins. The radiolabeled alcohol that is accumulated and trapped in acidic lysosomes may be detected by a radiology device, such as a PET/CT device.

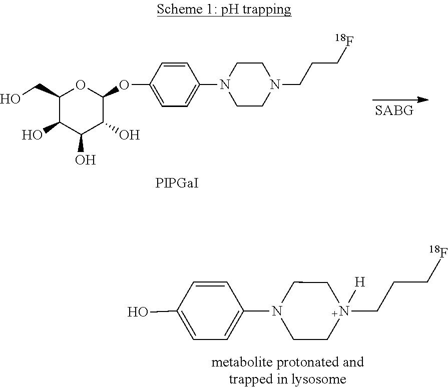

Scheme 2 shows a second approach. In said case, after senescence associated beta-galactosidase (SABG) mediated cleavage of the glucosidase moiety, the alcohol exhibits a structure, which decomposes to a quinone methide. The quinone methide reacts with amino residues, particularly, amino residues of cysteine and lysine, of amino acids of proteins that are present in the target cell (shown by an arrow) and forms a bond with the amino residues under formation of a secondary amine. In said secondary amine of the so-called quinone methide binding, one residue is (still) linked to the protein and the other residue is connected with a residue of the former radioactive tracer, which carries the radioactive label. The radiolabeled alcohol that is bond to cellular proteins may be detected by a radiology device, such as a PET/CT device.

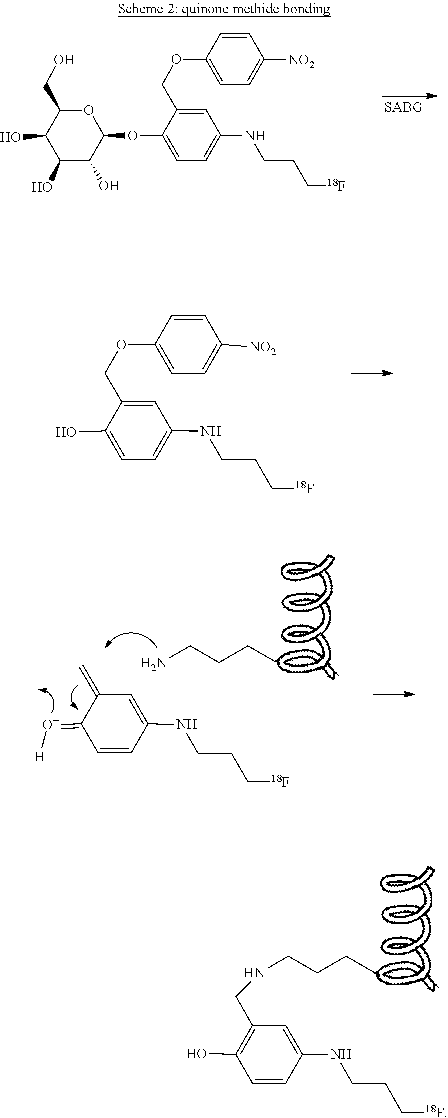

The compounds of the present invention may be prepared as schematically shown hereinafter.

Scheme 3 shows a first part of the precursor synthesis according to quinone methide bonding:

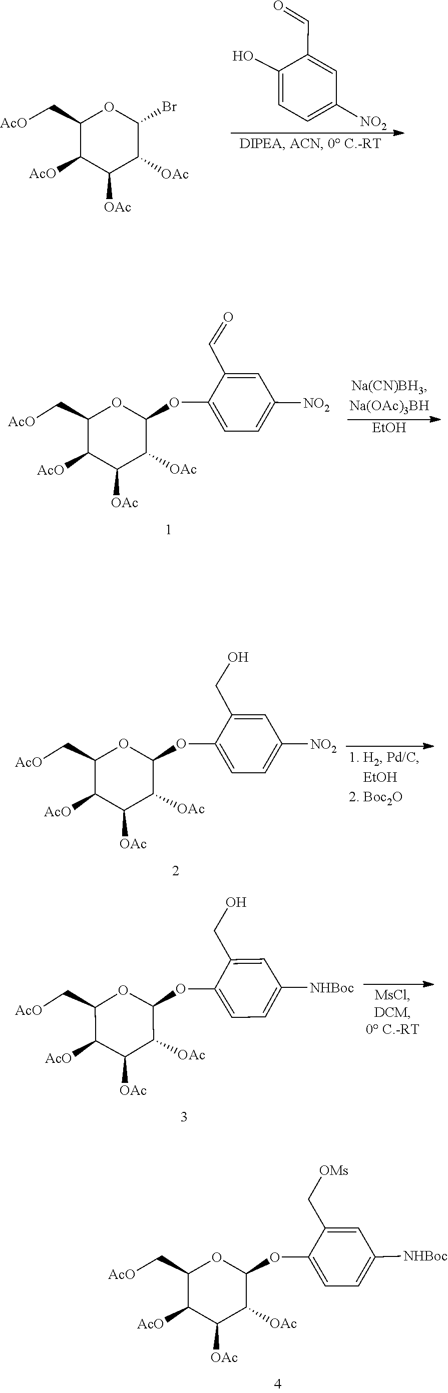

In a first step, the hexose precursor carrying a bromine at the glycosidic bonding position and acetylated hydroxyl groups is reacted with a hydroxy-benzaldehyde, preferably 2-hydroxy-benzaldehyde, and a nitro group, such as 2-hydroxy-5-nitrobenzaldehyde forming a glycoside 1 The aldehyde group is converted to an alcohol 3, the nitro group is protected 3, e.g., with a tert-butyloxycarbonyl (Boc) protecting group, and the alcohol is provided 4 with, e.g., a methylsulfonyl (MS) group. Thereby, the alcohol is converted into a suitable leaving group, creating an alkylating agent. This allows for labeling with the aryl leaving group needed for the self-immolating reaction and generation of the quinone methide.

Scheme 4 shows a second part of the precursor synthesis according to quinone methide bonding:

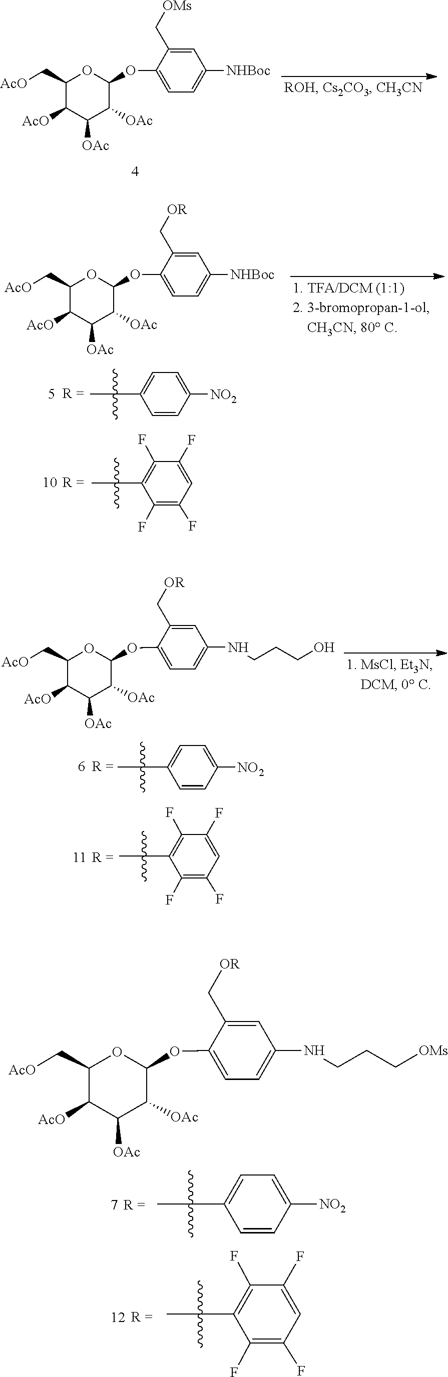

The MS activated hydroxyl group of the Boc and MS substituted precursor 4 is converted to an ether 5, 10. In the next step the Boc protection group is converted to a secondary amine having a hydroxyl moiety 6, 11, which is again activated by converting into a mesyl group 7, 12 to allow for late-stage radio-fluorination.

Scheme 5 shows a first part of the cold standard synthesis:

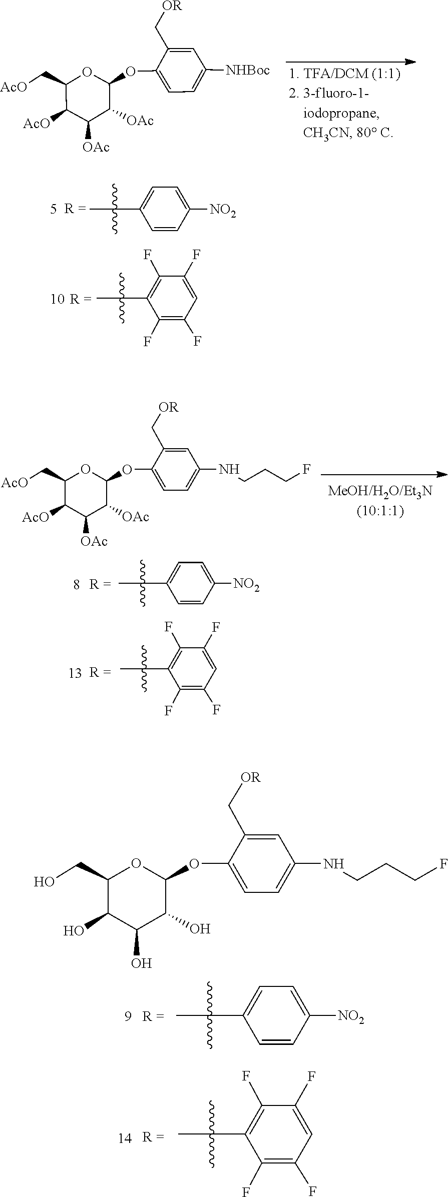

The Boc protection group of the ether 5, 10 of scheme 4 is converted to a secondary amine having a fluorine moiety. Acyl groups of the hexose moiety are subsequently removed to yield the cold standard 9, 14.

Scheme 6 shows PIPGal synthesis:

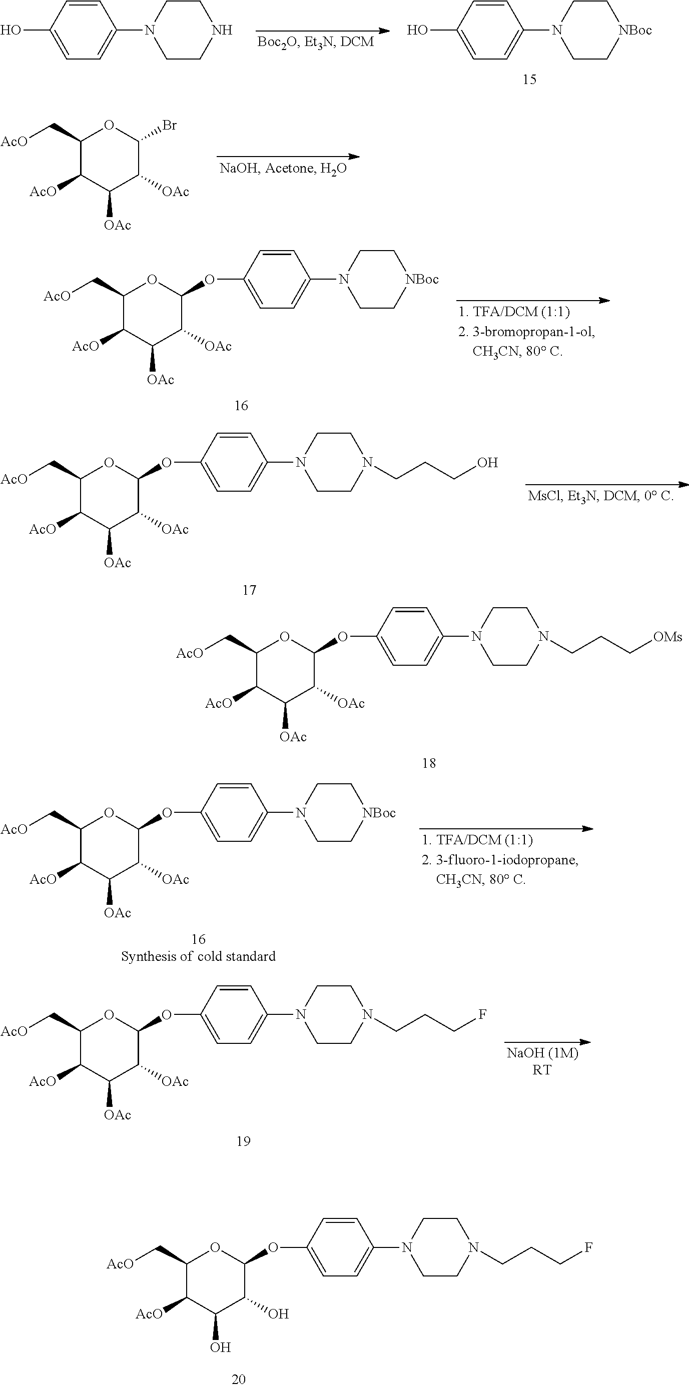

In a first step, the brominated and acetylated hexose derivative, such as acetobromogalactose, is converted with a protected 4-(piperazin-1-yl)phenol derivative 15 and subsequently deprotected to yield the Boc protected glycoside 16 that is converted to the mesylate 18. The cold standard may be synthesized by reacting the acetylated hexose derivative, such as acetobromogalactose, with a protected 4-(piperazin-1-yl)phenol derivative 15, which is, then alkylated 19 and deprotected 20.

Scheme 7 shows the synthesis of the TFPBGal precursor:

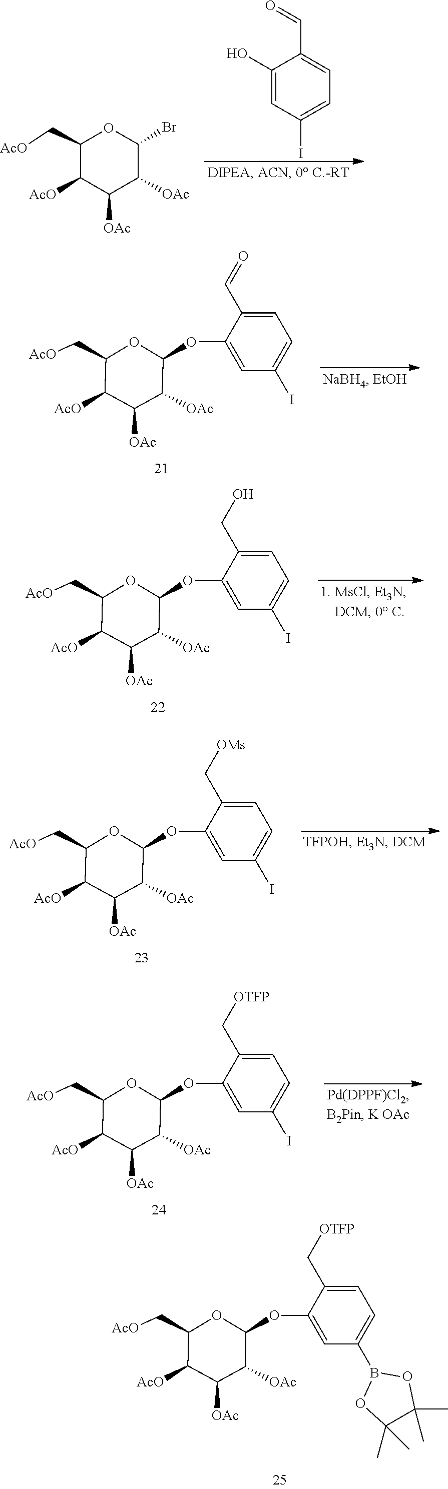

As may be derived from scheme 7, the hexose derivative is converted with a (iodine or bromine) substituted ortho salicylaldehyde to yield the compound 21, which is converted with NaBH₄ to yield the alcohol 22. The alcohol is mesylated 23 and converted to tetrafluorophenylether 24. The iodine or bromine substitution is then reacted to yield compound 25.

Scheme 8 shows the synthesis of the TFPBGal cold standard:

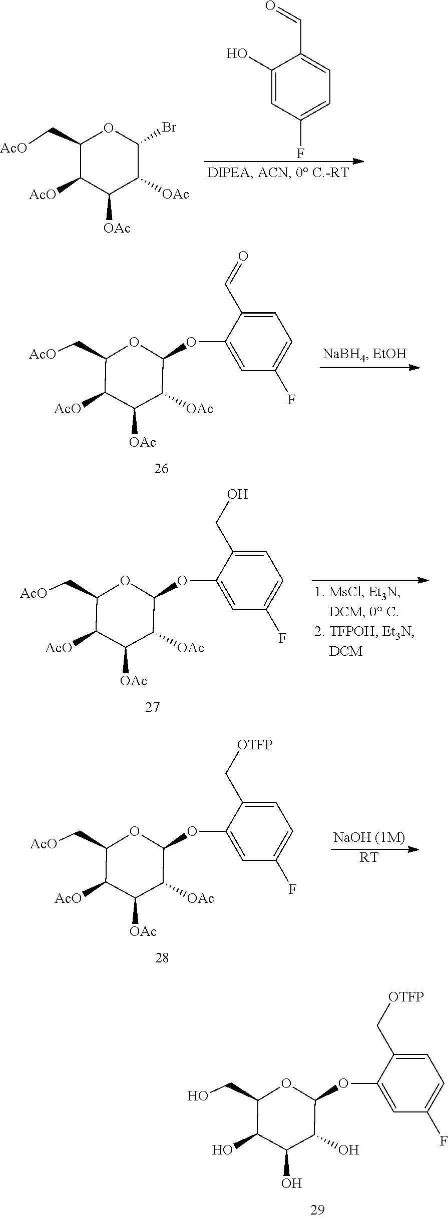

The hexose derivative is reacted with fluorine substituted ortho salicylaldehyde to yield the compound 26, which is converted with NaBH₄ to yield the alcohol 27. The alcohol is converted to the tetrafluorophenylether 28 and de-acetylated.

The compounds of the present invention show a valuable range of pharmalogical effects which could not have been predicted. They are capable of marking senescent cells in vitro and in vivo. Particularly, in vivo marking is made to such an extent that senescent cells may be unambiguously identified in course of a surgical procedure which in turn allows targeted elimination of such cells, particularly cancer cells.

It is to be understood that the above description is intended to be illustrative only and not restrictive. Many embodiments will be apparent to those skilled in the art upon reviewing the above description. By way of example, the invention has been described preliminarily with reference to synthesis as well as diagnosis of tracer 9. It should be clear that all kinds of suitable detectable labels and therapeutic residues may be synthesized and attached to the present compounds. The scope of the invention should, therefore, be determined not with reference to the above description, but should instead be determined with reference to the appended claims, along with the full scope of equivalents to which such claims are entitled.

The percentage data in the following tests and examples are, unless indicated otherwise, percentages by weight; parts are parts by weight. Solvent ratios, dilution ratios and concentration data of liquid/liquid solutions are based in each case on volume. The statement “w/v” means “weight/volume”. Thus, for example, “10% w/v” means: 100 ml of solution or suspension contain 10 g of substance.

### EXAMPLES

### General Methods

[¹⁸F]Fluoride was produced by a PETtrace 890 cyclotron (GE Healthcare, Uppsala, Sweden) and delivered directly into the module. The fluoride was trapped on the QMA cartridge, eluted into the reactor with either the Kryptofix or TBAOTf solution, after which the solvent was evaporated at elevated temperature.

For standard nucleophilic radiofluorination, Sep-Pak Plus light QMA carb cartridges were conditioned with NaHCO₃ (10 mL), air (10 mL), H₂O (10 mL), air (10 mL). For copper-mediated radiofluorination (TFPBGal), Sep-Pak QMA carb light cartridge was conditioned with 10 ml KOTf₂ solution (90 mg/ml), air (10 mL), H₂O (10 mL) and air (10 mL). Sep-Pak Light C18 cartridges were conditioned with EtOH (10 mL) and H₂O (10 mL). Alumina N cartridge were conditioned with H₂O (5 mL).

Kryptofix eluent used for all nucleophilic radiofluorinations comprised Kryptofix (9.5 mg), K₂CO₃ (1.7 mg), H₂O (80 μl) and CH₃CN (1.92 mL). The elution of fluoride for copper-mediated radiofluorinations utilized TBAOTf (5 mg) in MeOH (1 mL).

All tracers were verified using radio-HPLC using the retention of their respective reference compound to confirm their identity. Analytical radio-HPLC data were collected using an Agilent HPLC (1260 Infinity series with an automated sample injector) coupled to an in-line radiation detector [NaI(TI)].

Automated Synthesis of [¹⁸F]9 ([¹⁸F]ADNO₂BGal) and [¹⁸F]14 ([¹⁸F]ADTFPBGal)

The selected precursor (7 or 12, 3.00 mg, 4.13 or 3.98 μmol respectively) was dissolved in DMF (500 μL). Addition of the precursor solution and heating at 150° C. for 10 min afforded the acetylated product, which was deprotected by addition of NaOH aq. 0.1 M. The reaction mixture was neutralized by a NaH₂PO₄ aq. 0.1 M solution and transferred onto the preparative HPLC. Product purification was performed on an Elixys Pure/Form module module (Sofie Bioscience, Culver City, California, USA) equipped with a Luna 5 μm C18 (2) 100 Å 250×10 mm column (Phenomenex, Torrance, California, USA) applying a step gradient at 6 mL/min Step gradient for [¹⁸F]9: 0.0 to 7.0 min 10% B, 7.0 to 13.0 min 60% B, 13.0 to 15.0 min 100% B; solvent A: H₂O, solvent B: MeCN; retention time=11.5 min. Step gradient for [¹⁸F]14: 0.0 to 6.5 min 10% B, 6.5 to 18.0 min 40% B, 18.0 to 20.0 min 100% B; solvent A: H₂O, solvent B: MeCN; retention time=16.5 min.

The product peak was collected, diluted with water (50.0 mL), trapped on the C18 cartridge, eluted in the product vial with EtOH (0.50 mL), and formulated with a phosphate-buffered saline solution (4.50 mL). Quality control was performed via analytical HPLC. Quality control was performed via analytical radio-HPLC, equipped with a Luna 5 μm C18 (2) 100 Å 250×4.6 mm column (Phenomenex, Torrance, California, USA) using the following gradient: 0-2 min (5% B), 2-17 min (5% to 100% B), 17-23 min (100% B), 23-28 min (100% to 5% B), 28-29 min (5% B); solvent A: 0.1% TFA in H₂O, solvent B: MeCN; 1 mL/min. Retention time for [¹⁸F]6≈10.7 min; retention time for [¹⁸F]11≈11.0 min.

Automated Synthesis of [¹⁸F]PIPGal [¹⁸F]20 and [¹⁸F]TFPBGal [¹⁸F]29

Automated ¹⁸F tracer syntheses were performed on a either a GE FX N Pro synthesis module (GE Healthcare, Muenster, Germany) running the TRACERIab (GE) control and user-interface software.

The radiochemical reaction performance was monitored using radio-TLC on 0.20 mm Polygram SIL G/UV₂₅₄ (silica gel 60) TLC plates. Radio-TLC plates were developed with an appropriate running buffer/solvent mixture. Radio-TLC data were acquired using a Cyclone Plus storage phosphor imaging system (PerkinElmer, Waltham, Massachusetts, USA).

Automated Synthesis of [¹⁸F]PIPGal (FX N pro)

Precursor 18 (10 mg) dissolved in DMSO (500 μl) was added to the reactor containing the azeotropically dried fluoride and heated to 150° C. for 15 min. The reactor was then cooled to 40° C., after which 0.1 M NaOH (1.0 mL) was added. The reaction mixture was stirred for 2 minutes at 40° C., after which 0.1 M NaH2PO4 (2.0 mL) was added. Purification using semi preparative HPLC (Luna 5 μm C18 (2) 100 Å 250×4.6 mm, Phenomenex) using 10% EtOH in phosphate buffered saline (PBS) at 6 ml/min. The product was collected in accordance with its radioactive signal (13-14 minutes) and delivered to a product vial. Quality control was performed by HPLC using an ABZ+ column (90% 25 mM Ammonium formate in CH3CN at 1 mL/min. Radio-TLC (35% acetone, 35% 1-butanol, 10% acetic acid und 20% H2O) was performed to assess purity.

Automated Synthesis of [¹⁸F]TFPBGal (FX N pro)

To the dried fluoride was added a solution containing precursor 25 (11 mg), Pyridine (3.26 μl) and Cu(OTf)₂ (3.7 mg) in 700 μl DMA. The reaction was heated at 120° C. for 20 min, after which it was cooled to 40° C. The deprotection was performed by the additions of 2M NaOH (1 ml), reacting for 10 minutes at 40° C. To the reaction mixture was then added 0.3M HCl (500 μl). This mixture was passed through an Alumina N cartridge and diluted with a solution containing 4M HCl (500 μl) and HPLC-Eluent (2 ml), before purification using semi-preparative HPLC (Luna 5 μm C18 (2) 100 Å 250×4.6 mm, Phenomenex) using an eluent comprising 35% MeCN in 0.1% TFA in H₂O at 6 ml/min. The product was collected in accordance with its radioactive signal (10-15 minutes) and delivered to a dilution reservoir containing water (50 mL). The solution was then passed over a SepPak C18 cartridge to immobilize the product, which was in turn washed with water (5.0 mL) and eluted with EtOH (0.5 mL) into a product vial containing with PBS (4.5 ml). Radio-TLC using EtOAc was performed to assess purity.

### Biology Experimental Methods

Cells were cultured in a humidified incubator (Binder, Tuttlingen, Germany) at 37° C. and 5% CO₂. Fetal bovine serum (FBS, Sigma of Merck) was heat-inactivated for 30 min at 57° C. for cell culture supplementation. For in vitro uptake experiments, the cells were rinsed with DPBS, harvested with a cell scraper and finally centrifuged. The cell pellet was then washed with DPPS and resuspended in DPBS, before being counted. A suspension of 1×10⁶ cells in 1 ml DPBS was incubated with 0.74 MBq of tracer at 37° C. and 5% CO₂ for 40 min. After the designated incubation time, the suspension was centrifuged. The cell pellet was transferred into a fresh gamma counter tube (Sarstedt), washed twice with 1 ml DPBS and resuspended in 1 ml DPBS. The uptake was measured with a 2480 Wizard² automated gamma counter (Perkin Elmer).

X-gal staining was performed to confirm senescence in vitro. Thus, cells were fixed with 0.25% glutaraldehyde (Sigma of Merck) and 2% formaldehyde (Sigma of Merck) in DPBS solution at room temperature for 15 min. The cells were then washed twice with 1 mM magnesium chloride (MgCl₂, Sigma of Merck) in DPBS solution (pH 6.0) and incubated at 37° C. Freshly prepared, prewarmed and filtered staining solution was added. The staining solution comprised 1 mg/ml 5-Bromo-4-chloro-3-indolyl-beta-D-galactopyranoside (X-gal, PEQLAB/VWR of Avantor), 5 mM potassium hexacyanoferrate(III) [K₃Fe(CN)₆, Sigma of Merck], and 5 mM potassium hexacyanoferrate(II) [K₄Fe(CN)₆, Merck] in 1 mM MgCl₂/DPBS solution at pH 6.0. The samples were wrapped in aluminum foil to protect them from light and to prevent evaporation. The samples were then washed twice with 1 mM MgCl₂/DPBS solution (pH 6.0), after which the they were examined with a light microscope (Axiovert 200 equipped with AxioCam and AxioVision, Carl Zeiss, Oberkochen, Germany).

Animal procedures were approved by the local authorities (Regierungspraesidium Tuebingen, Tuebingen, Germany) and conducted according to the German Animal Welfare Act. The mice used were between 6 and 7 weeks of age. Tumors were implanted by injecting 0.5×10⁶ cells s.c. into the right flank of SCID beige mice. When most tumors reached 100 mm³ (˜3 weeks post inoculation), the animals were randomized into two groups. The groups received 25 mg/kg/d CX-5461 (AdooQ BioScience, Irvine, CA, USA) or vehicle [50 mM monosodium phosphate (Sigma of Merck KGaA, Darmstadt, Germany), pH 4.5]i.p. for five consecutive days.

Anatomical imaging was conducted on a 7 Tesla small animal MRI scanner with a rat whole body coil and Paravision® software (Bruker, Billerica, MA, USA). The animals were then transferred to the PET scanner without moving the animal to ensure co-registration. The animals received 12±1 MBq tracer (determined by a CRC 15-R dose calibrator) was injected through a tail vein catheter, which was subsequently flushed with 50 μl heparinized saline solution. The transmission scan using an external ⁵⁷Co point source was performed for the attenuation correction. The static emission scan was acquired 50-60 min after tracer injection.

The list mode raw file was histogrammed and reconstructed with OSEM3D/SP-MAP algorithm implemented in Inveon Acquisition Workplace. The reconstructed PET and corresponding anatomical MRI images were imported into the Inveon Research Workplace software (Siemens) and manually co-registered. Volumes of interest were drawn manually based on the anatomical reference to extract values (Bq/ml) from the respective PET image. The tracer uptake was calculated as the percentage of the injected dose per cubic centimeter (% ID/cc).

For autoradiography, the tumors were embedded in the optimal cutting temperature compound (Tissue-Tek® O.C.T., Sakura, Alphen aan den Rijn, Netherlands) and frozen at −20° C. in the cryotome (Leica, Wetzlar, Germany). Cryosections (20 μm) were then attached to microscope slides (SuperFrost® Plus, R. Langenbrinck, Emmendingen, Germany). The slides were exposed overnight to a phosphor screen (Molecular Dynamics of GE Healthcare). The screen was then scanned by the phosphorimager (Storm 840, Amersham of GE Healthcare). The signal intensities of sections were normalized to each other based on decay corrected animal receiving dose with the open-source software ImageJ (National Institute of Health, Bethesda, MD, USA).

Hematoxylin and eosin (H&E) staining was accomplished for the autoradiography sections with an automated system by the staff at the Department of Dermatology (University Hospital Tuebingen, Germany).

Immunohistochemistry (IHC) was conducted with the automated immunostainer (Ventana Medical Systems of Roche) according to the manufacturer's protocol by the staff at the Department of Pathology (University Hospital Tuebingen, Germany).

### Example 1

Example 1 discloses the precursor synthesis of compounds according to reaction scheme 3.

### Synthesis of Compound 1

### (2R,3S,4S,5R,6S)-2-(acetoxymethyl)-6-(2-formyl-4-nitrophenoxy)tetrahydro-2H-pyran-3,4,5-triyl triacetate, 1

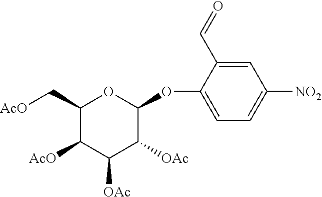 [SMILES: CC(=O)OC[C@H]1O[C@@H](Oc2ccc([N+](=O)[O-])cc2C=O)[C@H](OC(C)=O)[C@@H](OC(C)=O)[C@H]1OC(C)=O]

Diisopropylethyl amine (7 mL) was added dropwise to an ice cooled solution of acetobromogalactose (5.0 g, 12.2 mmol) and 2-hydroxy-5-nitrobenzaldehyde (2.2 g, 13.4 mmol) in CH₃CN (50 mL), under an argon atmosphere. The mixture was left to stir overnight while warming slowly to room temperature. After concentrating the mixture under reduced pressure, resulting residue was diluted with EtOAc (200 mL), after which it was washed successively with 5% citric acid (3×100 mL), water (2×100 mL), saturated aqueous NaHCO₃ (2×100 mL) and brine (100 mL). The organic layer was then dried over Na₂SO₄, after which the volume reduced to around 30 mL under reduced pressure. The mixture was allowed to crystalize at −20° C. overnight. The resulting crystals were collected and the mother liquor was reduced to afford a second crop of crystals. The aforementioned colorless crystals were collecting by filtration and dried (4.9 g, 81.1%). R_{f}: 0.5 (PE/EtOAc 1:1); ¹H NMR (600 MHz, CDCl₃) δ 10.30 (s, 1H), 8.66 (d, J=2.9 Hz, 1H), 8.38 (dd, J=9.2, 2.9 Hz, 1H), 7.34-7.18 (m, 1H), 5.58 (dd, J=10.5, 7.8 Hz, 1H), 5.49 (d, J=3.5 Hz, 1H), 5.31 (d, J=7.9 Hz, 1H), 5.17 (dd, J=10.5, 3.4 Hz, 1H), 4.22-4.14 (m, 3H), 2.18 (s, 3H), 2.05 (s, 3H), 2.04 (s, 3H), 2.00 (s, 4H).

### Synthesis of Compound 2

### (2R,3S,4S,5R,6S)-2-(acetoxymethyl)-6-(2-(hydroxymethyl)-4-nitrophenoxy)tetrahydro-2H-pyran-3,4,5-triyl triacetate, 2

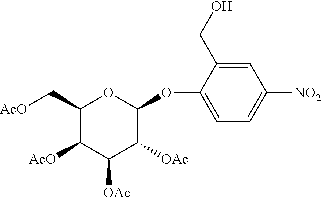 [SMILES: CC(=O)OC[C@H]1O[C@@H](Oc2ccc([N+](=O)[O-])cc2CO)[C@H](OC(C)=O)[C@@H](OC(C)=O)[C@H]1OC(C)=O]

The aldehyde 1 (2.1 g, 4.2 mmol) was dissolved in EtOH (40 mL). To this solution was added Na(CN)BH₃ (2.7 g, 12.85 mmol) and Na(OAc)₃BH (0.26 g, 3.4 mmol) carefully in small portions. The reaction mixture was allowed to stir at room temperature for 1 hour, after which water (30 mL) and EtOAc (300 mL) were added successively. The organic layer was washed with water (2×100 mL) and brine (100 mL), after which it was dried over Na₂SO₄ and concentrated under reduced pressure. The product was allowed to crystalize overnight at −20° C., with the mother liquor affording a second crop of crystals after its volume was reduced. The afforded solids were collected by filtration and dried under reduced pressure (1.79 g, 85%). R_{f}: 0.45 (PE/EtOAc 1:1); ¹H NMR (600 MHz, CDCl₃) b 8.29 (d, J=2.8 Hz, 1H), 8.16 (dd, J=9.0, 2.8 Hz, 1H), 7.07 (d, J=9.0 Hz, 1H), 5.53 (dd, J=10.5, 7.8 Hz, 1H), 5.49 (d, J=3.4 Hz, 1H), 5.20-5.15 (m, 2H), 4.67 (d, J=1.9 Hz, 2H), 4.22 (td, J=9.3, 5.3 Hz, 1H), 4.19-4.13 (m, 2H), 2.20 (s, 3H), 2.10 (s, 3H), 2.07 (s, 3H), 2.03 (s, 3H).

### Synthesis of Compound 3

### (2R,3S,4S,5R,6S)-2-(acetoxymethyl)-6-(4-((tert-butoxycarbonyl)amino)-2-(hydroxymethyl)phenoxy)tetrahydro-2H-pyran-3,4,5-triyl triacetate, 3

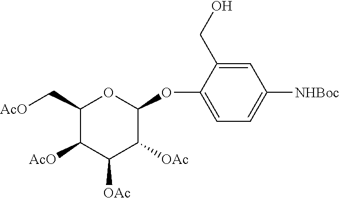 [SMILES: CC(=O)OC[C@H]1O[C@@H](Oc2ccc(NC(=O)OC(C)(C)C)cc2CO)[C@H](OC(C)=O)[C@@H](OC(C)=O)[C@H]1OC(C)=O]

To a suspension of 5% Pd/C (0.74 g) in EtOAc was added compound 2 (2.3 g, 4.5 mmol) in EtOAc (5 mL). The solution was degassed and stirred for 4 hours under a hydrogen atmosphere. After complete consumption of the starting material as revealed by TLC, the atmosphere was exchanged for argon and Boc₂O (1.5 g, 4.5 mmol) in EtOAc (5 mL) was added through a septum. The reaction was stirred overnight at room temperature, after which it was filtered through celite. After removing the solvents under reduced pressure, the resulting residue was purified using silica gel flash chromatography (5-100% EtOAc in PE), affording the product as a colorless oil (1.9 g, 73%). ¹H NMR (600 MHz, CDCl₃) δ 7.30 (s, 1H), 5.48 (dd, J=10.5, 7.9 Hz, 1H), 5.44 (dd, J=3.5, 1.1 Hz, 1H), 5.11 (dd, J=10.5, 3.4 Hz, 1H), 4.98 (d, J=8.0 Hz, 1H), 4.23-4.13 (m, 2H), 4.02 (ddd, J=7.2, 5.9, 1.2 Hz, 1H), 2.04 (s, 4H), 6.96 (d, J=8.7 Hz, 1H), 6.46 (s, 1H), 4.64 (d, J=12.9 Hz, 1H), 4.51 (d, J=12.9 Hz, 1H), 2.19 (s, 3H), 2.10 (s, 3H), 2.01 (s, 3H), 1.50 (s, 9H).

### Example 2

Example 2 discloses the precursor synthesis of compounds according to reaction scheme I.

### Synthesis of Compound 4

### (2R,3S,4S,5R,6S)-2-(acetoxymethyl)-6-(4-((tert-butoxycarbonyl)amino)-2-(((methylsulfonyl)oxy)methyl)phenoxy)tetrahydro-2H-pyran-3,4,5-triyl triacetate, 4

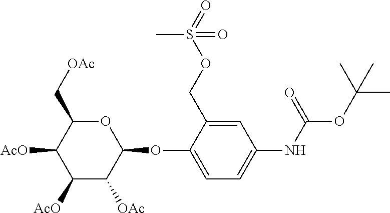 [SMILES: CC(=O)OC[C@H]1O[C@@H](Oc2ccc(NC(=O)OC(C)(C)C)cc2COS(C)(=O)=O)[C@H](OC(C)=O)[C@@H](OC(C)=O)[C@H]1OC(C)=O]

A solution of 3 (3.44 g, 6.04 mmol) in DCM (180 mL) under argon atmosphere was cooled down to 0° C. and Et₃N (3.00 mL, 18.1 mmol) was added. MsCl (0.51 mL, 6.64 mmol) was added dropwise and the mixture was stirred for 45 min at 0° C. The reaction was quenched with aq. NaHCO₃, extracted with DCM and subsequently washed with aqueous solutions of citric acid and NaHCO₃. The organic phase was dried over MgSO₄, evaporated and purified by flash chromatography (PE/EtOAc 5% to 100% B) to afford the product (3.03 g, 77%). R_{f}: 0.62 (PE/EtOAc 1:2). ¹H NMR (600 MHz, DMSO-d₆) δ 9.34 (s, 1H), 7.55 (s, 1H), 7.42 (t, J=8.4 Hz, 1H), 7.06 (dd, J=9.0, 2.7 Hz, 1H), 5.38 (dd, J=7.9, 2.6 Hz, 1H), 5.35 (d, J=3.5 Hz, 1H), 5.29 (dd, J=10.6, 3.4 Hz, 1H), 5.23 (td, J=10.3, 7.7 Hz, 1H), 5.10 (qd, J=11.6, 2.7 Hz, 2H), 4.42 (t, J=6.5 Hz, 1H), 4.15-4.07 (m, 2H), 3.19 (s, 3H), 2.15 (s, 3H), 2.07 (s, 3H), 2.01 (s, 3H), 1.95 (s, 3H), 1.46 (s, 12H).

### Synthesis of Compound 5

### (2R,3S,4S,5R,6S)-2-(acetoxymethyl)-6-(4-((tert-butoxycarbonyl)amino)-2-((4-nitrophenoxy)methyl)phenoxy)tetrahydro-2H-pyran-3,4,5-triyl triacetate, 5

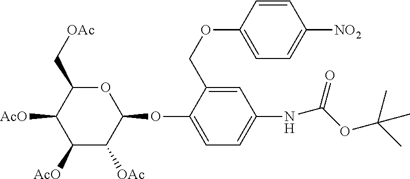 [SMILES: CC(=O)OC[C@H]1O[C@@H](Oc2ccc(NC(=O)OC(C)(C)C)cc2COc2ccc([N+](=O)[O-])cc2)[C@H](OC(C)=O)[C@@H](OC(C)=O)[C@H]1OC(C)=O]

A mixture of 4 (1.57 g, 2.43 mmol) and 4-nitrophenol (670 mg, 4.85 mmol) was dissolved in MeCN under argon atmosphere. Cs₂CO₃ (1.74 g, 5.33 mmol) was added and the mixture was stirred overnight at room temperature. The reaction mixture was diluted with water and extracted with EtOAc. The organic phase was dried over MgSO₄, evaporated and purified by flash chromatography to afford the product (1.58 g, 94%). ¹H NMR (600 MHz, CDCl₃) δ 8.22 (d, J=9.2 Hz, 2H), 7.41 (d, J=2.6 Hz, 1H), 7.36 (d, J=8.9 Hz, 1H), 7.05-7.02 (m, 2H), 7.00 (d, J=8.9 Hz, 1H), 5.50 (dd, J=10.5, 7.9 Hz, 1H), 5.47 (dd, J=3.4, 1.1 Hz, 1H), 5.12 (dd, J=10.5, 3.5 Hz, 1H), 5.10 (d, J=3.3 Hz, 2H), 5.03 (d, J=7.9 Hz, 1H), 4.22 (dd, J=11.3, 7.0 Hz, 1H), 4.16 (t, J=5.7 Hz, 1H), 4.09-4.05 (m, 1H), 2.19 (s, 3H), 2.06 (s, 3H), 2.01 (s, 3H), 1.95 (s, 3H), 1.50 (s, 9H).

### Synthesis of Compound 6

### (2R,3S,4S,5R,6S)-2-(acetoxymethyl)-6-(4-((3-hydroxypropyl)amino)-2-((4-nitrophenoxy)methyl)phenoxy)tetrahydro-2H-pyran-3,4,5-triyl triacetate, 6

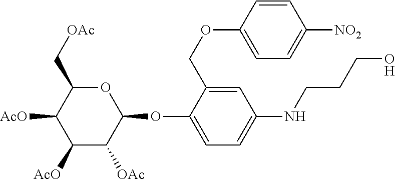 [SMILES: CC(=O)OC[C@H]1O[C@@H](Oc2ccc(NCCCO)cc2COc2ccc([N+](=O)[O-])cc2)[C@H](OC(C)=O)[C@@H](OC(C)=O)[C@H]1OC(C)=O]

A solution of 5 (265 mg, 0.38 mmol) in DCM/TFA 1:1 v/v (5.3 mL, 5.3 mL) was stirred for 15 min at room temperature. The solvent was evaporated under vacuum and the residue dissolved in dry MeCN (8.00 mL) under argon atmosphere. NaI (23.5 mg, 0.16 mmol), Et₃N (0.21 mL, 1.49 mmol) and 3-bromo-1-propanol (0.04 mL, 0.45 mmol) were added. The mixture was heated overnight at 80° C. The reaction mixture was diluted with water and extracted with EtOAc. The organic phase was dried over MgSO₄, evaporated and purified by flash chromatography (PE/EtOAc 16% to 100% B) to afford the product (80.0 mg, 32%). ¹H NMR (600 MHz, CDCl₃) δ 8.23-8.19 (m, 2H), 7.07-7.03 (m, 2H), 6.98 (d, J=8.8 Hz, 1H), 6.96-6.93 (m, 1H), 6.80 (d, J=8.6 Hz, 1H), 5.48 (dd, J=10.5, 7.9 Hz, 2H), 5.46 (dd, J=3.5, 1.1 Hz, 1H), 5.14-5.11 (m, 1H), 5.10 (s, 2H), 4.98 (d, J=7.9 Hz, 1H), 4.18 (ddd, J=41.1, 11.6, 6.2 Hz, 3H), 4.05 (td, J=6.6, 1.1 Hz, 1H), 3.81 (t, J=5.7 Hz, 2H), 3.29 (t, J=6.4 Hz, 2H), 2.19 (s, 3H), 2.06 (s, 4H), 2.01 (s, 3H), 1.97 (s, 3H), 1.89 (p, J=6.1 Hz, 2H).

### Synthesis of Compound 7

### (2R,3S,4S,5R,6S)-2-(acetoxymethyl)-6-(4-((3-((methylsulfonyl)oxy)propyl)amino)-2-((4-nitrophenoxy)methyl)phenoxy)tetrahydro-2H-pyran-3,4,5-triyl triacetate, 7

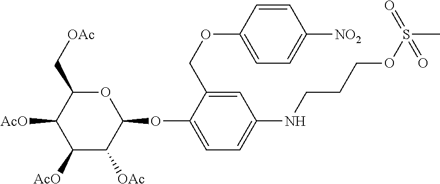 [SMILES: CC(=O)OC[C@H]1O[C@@H](Oc2ccc(NCCCOS(C)(=O)=O)cc2COc2ccc([N+](=O)[O-])cc2)[C@H](OC(C)=O)[C@@H](OC(C)=O)[C@H]1OC(C)=O]

A solution of 6 (80.0 mg, 0.12 mmol) in dry DCM (6.00 mL) under argon atmosphere was cooled down to 0° C. and Et₃N (0.05 mL, 0.37 mmol) was added. MsCl (0.01 mL, 0.12 mmol) was added dropwise at and the mixture was stirred for 1 hour at 0° C. The reaction mixture was diluted with water and extracted with DCM. The organic phase was dried over MgSO₄, evaporated and purified by flash chromatography (PE/EtOAc 16% to 100% B) to afford the product (59.0 mg, 66%). ¹H NMR (600 MHz, DMSO-d₆) δ 8.26-8.20 (m, 2H), 7.20-7.13 (m, 2H), 6.94 (d, J=8.8 Hz, 1H), 6.64 (d, J=2.8 Hz, 1H), 6.56 (dd, J=8.9, 2.8 Hz, 1H), 5.56 (t, J=5.8 Hz, 1H), 5.32 (dd, J=3.5, 1.2 Hz, 1H), 5.26 (dd, J=10.1, 3.6 Hz, 1H), 5.22-5.14 (m, 2H), 5.06 (q, 2H), 4.36 (ddd, J=7.0, 5.3, 1.2 Hz, 1H), 4.28 (t, J=6.3 Hz, 2H), 4.13 (dd, J=11.3, 7.4 Hz, 1H), 4.06 (t, J=5.4 Hz, 1H), 3.16 (s, 3H), 3.06 (q, J=6.5 Hz, 2H), 2.14 (s, 3H), 2.01 (s, 3H), 1.92 (s, 3H), 1.92 (s, 3H), 1.89 (q, J=6.6 Hz, 2H).

### Example 3

Example 3 discloses the precursor synthesis of compounds according to reaction scheme 5.

### Synthesis of Compound 8

### (2R,3S,4S,5R,6S)-2-(acetoxymethyl)-6-(4-((3-fluoropropyl)amino)-2-((4-nitrophenoxy)methyl)phenoxy)tetrahydro-2H-pyran-3,4,5-triyl triacetate, 8

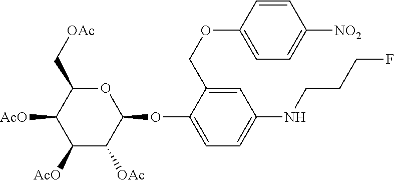 [SMILES: CC(=O)OC[C@H]1O[C@@H](Oc2ccc(NCCCF)cc2COc2ccc([N+](=O)[O-])cc2)[C@H](OC(C)=O)[C@@H](OC(C)=O)[C@H]1OC(C)=O]

A solution of 5 (140 mg, 0.20 mmol) in DCM/TFA 1:1 v/v (2.8 mL, 2.8 mL) was stirred for 15 min at room temperature. The solvent was evaporated under reduced pressure and the residue dissolved in dry MeCN (5.30 mL) under argon atmosphere. NaI (13.9 mg, 0.09 mmol), Et₃N (0.12 mL, 0.88 mmol) and 3-fluoro-1-iodopropane (0.03 mL, 0.26 mmol) were added. The mixture was heated overnight at 80° C. The reaction mixture was diluted with water and extracted with EtOAc. The organic phase was dried over MgSO₄, evaporated and purified by flash chromatography (PE/EtOAc 16% to 100% B) to afford the product (58.0 mg, 45%). ¹H NMR (600 MHz, CDCl₃) δ 8.21 (dt, J=9.2, 2.1 Hz, 2H), 7.05 (dt, J=9.3, 2.3 Hz, 2H), 6.97 (d, J=8.7 Hz, 1H), 6.88 (s, 2H), 6.74 (s, OH), 5.48 (dd, J=10.5, 8.0 Hz, 2H), 5.46 (d, J=3.5 Hz, 1H), 5.10 (s, 2H), 4.97 (d, J=7.9 Hz, 1H), 4.55 (dt, J=47.1, 5.5 Hz, 2H), 4.25-4.13 (m, 2H), 4.04 (t, J=6.7 Hz, 1H), 3.29 (t, J=6.9 Hz, 2H), 2.19 (s, 3H), 2.07 (s, 1H), 2.06 (s, 3H), 2.03 (s, 1H), 2.01 (s, 3H), 2.00 (s, 1H), 1.97 (s, 3H).

### Synthesis of Compound 9

### (2S,3R,4S,5R,6R)-2-(4-((3-fluoropropyl)amino)-2-((4-nitrophenoxy)methyl)phenoxy)-6-(hydroxymethyl)tetrahydro-2H-pyran-3,4,5-triol, 9

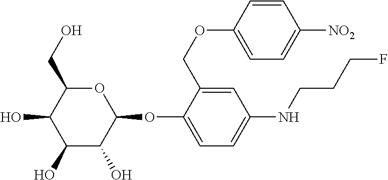 [SMILES: O=[N+]([O-])c1ccc(OCc2cc(NCCCF)ccc2O[C@@H]2O[C@H](CO)[C@H](O)[C@H](O)[C@H]2O)cc1]

A solution of 8 (20.0 mg, 0.02 mmol) in MeOH/H₂O/Et₃N 10:1:1 v/v (2.50 mL, 0.25 mL, 0.25 mL) was stirred for 90 min at room temperature. The solvent was evaporated under reduced pressure and the residue was purified by flash chromatography (DCM/MeOH 2% to 20% B) to afford the product (5.00 mg, 67%). ¹H NMR (600 MHz, DMSO-d₆) δ 8.20 (dt, J=9.3, 2.2 Hz, 2H), 7.21 (dt, J=9.4, 2.3 Hz, 2H), 7.00 (d, J=8.8 Hz, 1H), 6.58 (d, J=2.9 Hz, 1H), 6.49 (dd, J=8.8, 2.8 Hz, 1H), 5.42 (t, J=5.8 Hz, 1H), 5.29 (s, 2H), 5.23 (s, 1H), 4.83 (s, 1H), 4.65 (s, 1H), 4.56-4.52 (m, 2H), 4.49 (s, 1H), 4.47 (t, J=5.9 Hz, 1H), 3.68 (s, 1H), 3.02 (td, J=5.6, 1.5 Hz, 2H), 2.61 (p, J=1.9 Hz, 1H), 1.87 (s, 2H).

### Synthesis of Compound 10

### (2R,3S,4S,5R,6S)-2-(acetoxymethyl)-6-(4-((tert-butoxycarbonyl)amino)-2-((2,3,5,6-tetrafluorophenoxy)methyl)phenoxy)tetrahydro-2H-pyran-3,4,5-triyl triacetate, 10

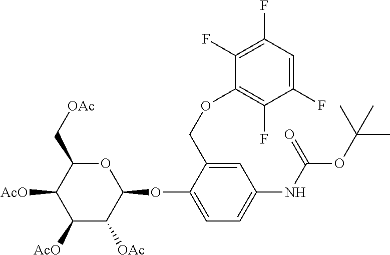 [SMILES: CC(=O)OC[C@H]1O[C@@H](Oc2ccc(NC(=O)OC(C)(C)C)cc2COc2c(F)c(F)cc(F)c2F)[C@H](OC(C)=O)[C@@H](OC(C)=O)[C@H]1OC(C)=O]

A mixture of 4 (3.00 g, 4.63 mmol) and 2,3,5,6-tetrafluorophenol (1.54 g, 9.26 mmol) was dissolved in MeCN under argon atmosphere. Cs₂CO₃ (3.32 g, 10.2 mmol) was added and the mixture was stirred overnight at room temperature. The reaction mixture was diluted with water and extracted with EtOAc to afford the product (3.20 g, 96%). R_{f}: 0.63 (PE/EtOAc 1:1). ¹H NMR (600 MHz, DMSO-d₆) δ 9.30 (s, 1H), 7.64-7.54 (m, 1H), 7.52 (s, 1H), 7.40 (d, J=8.9 Hz, 1H), 7.03 (dd, J=9.2, 2.5 Hz, 1H), 5.36-5.31 (m, 2H), 5.26 (dt, J=10.5, 3.5 Hz, 1H), 5.23 (dd, J=11.5, 2.5 Hz, 1H), 5.15 (td, 1H), 5.09 (d, J=11.6 Hz, 1H), 4.41 (t, J=6.4 Hz, 1H), 4.13-4.06 (m, 2H), 2.15 (d, J=2.5 Hz, 3H), 2.01 (d, J=2.5 Hz, 3H), 1.96 (d, J=2.5 Hz, 3H), 1.94 (d, J=2.4 Hz, 3H), 1.45 (d, J=2.6 Hz, 9H).

### Synthesis of Compound 11

### (2R,3S,4S,5R,6S)-2-(acetoxymethyl)-6-(4-((3-hydroxypropyl)amino)-2-((2,3,5,6-tetrafluorophenoxy)methyl)phenoxy)tetrahydro-2H-pyran-3,4,5-triyl triacetate, 11

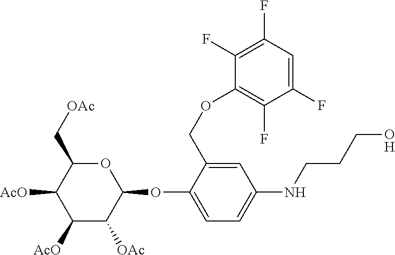 [SMILES: CC(=O)OC[C@H]1O[C@@H](Oc2ccc(NCCCO)cc2COc2c(F)c(F)cc(F)c2F)[C@H](OC(C)=O)[C@@H](OC(C)=O)[C@H]1OC(C)=O]

A solution of 10 (3.15 g, 4.39 mmol) in DCM/TFA 1:1 v/v (35 mL, 35 mL) was stirred for 30 min at room temperature. The solvent was evaporated under vacuum and the residue dissolved in dry MeCN (80.0 mL) under argon atmosphere. NaI (229 mg, 1.53 mmol), Et₃N (2.44 mL, 17.5 mmol) and 3-bromo-1-propanol (0.47 mL, 5.25 mmol) were added. The mixture was heated overnight at 80° C. The reaction mixture was diluted with water and extracted with EtOAc. The organic phase was dried over MgSO₄, evaporated and purified by flash chromatography (PE/EtOAc 5% to 100% B) to afford the product (244 mg, 8%). R_{f}: 0.18 (PE/EtOAc 1:2). ¹H NMR (600 MHz, DMSO-d₆) δ 7.59 (tt, J=10.8, 7.2 Hz, 1H), 7.03 (d, J=8.8 Hz, 1H), 6.98-6.83 (m, 2H), 5.32 (dd, J=3.6, 1.2 Hz, 1H), 5.30-5.25 (m, 2H), 5.24 (d, J=9.7 Hz, 1H), 5.15 (dd, J=10.4, 7.9 Hz, 1H), 5.10 (d, J=11.6 Hz, 1H), 4.38 (ddd, J=7.0, 5.5, 1.2 Hz, 1H), 4.13-4.06 (m, 2H), 3.48 (t, J=6.1 Hz, 2H), 3.11 (t, J=7.2 Hz, 2H), 2.15 (s, 3H), 2.01 (s, 3H), 1.97 (s, 3H), 1.94 (s, 3H), 1.70-1.64 (m, 2H).

### Synthesis of Compound 12

### (2R,3S,4S,5R,6S)-2-(acetoxymethyl)-6-(4-((3-((methylsulfonyl)oxy)propyl)amino)-2-((2,3,5,6-tetrafluorophenoxy)methyl)phenoxy)tetrahydro-2H-pyran-3,4,5-triyl triacetate, 12

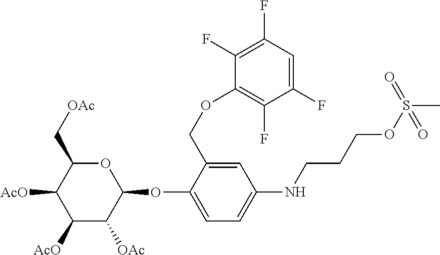 [SMILES: CC(=O)OC[C@H]1O[C@@H](Oc2ccc(NCCCOS(C)(=O)=O)cc2COc2c(F)c(F)cc(F)c2F)[C@H](OC(C)=O)[C@@H](OC(C)=O)[C@H]1OC(C)=O]

A solution of 11 (200 mg, 0.30 mmol) in dry DCM (40.0 mL) under argon atmosphere was cooled down to 0° C. and Et₃N (0.12 mL, 0.89 mmol) was added. MsCl (0.02 mL, 0.30 mmol) was added dropwise and the mixture was stirred for 1 hour at 0° C. The reaction mixture was diluted with water and extracted with DCM. The organic phase was dried over MgSO₄, evaporated and purified by flash chromatography (PE/EtOAc 10% to 100% B) to afford the product (89.0 mg, 40%). Rr: 0.52 (PE/EtOAc 1:3). ¹H NMR (600 MHz, DMSO-d₆) δ 7.58 (tt, J=10.8, 7.2 Hz, 1H), 6.91 (d, J=8.8 Hz, 1H), 6.62 (d, J=2.8 Hz, 1H), 6.57 (dd, J=8.9, 2.9 Hz, 1H), 5.56 (t, J=5.7 Hz, 1H), 5.30 (dd, J=3.6, 1.1 Hz, 1H), 5.26-5.20 (m, 2H), 5.16 (d, J=8.0 Hz, 1H), 5.12 (dd, J=10.1, 8.0 Hz, 1H), 5.07 (d, J=11.3 Hz, 1H), 4.34 (ddd, J=7.0, 5.5, 1.2 Hz, 1H), 4.29 (t, J=6.3 Hz, 2H), 4.13-4.05 (m, 2H), 3.16 (s, 3H), 3.06 (q, J=6.5 Hz, 2H), 2.15 (s, 3H), 2.00 (s, 3H), 1.97 (s, 3H), 1.93 (s, 3H), 1.90 (p, J=6.6 Hz, 2H).

### Synthesis of Compound 13

### (2R,3S,4S,5R,6S)-2-(acetoxymethyl)-6-(4-((3-fluoropropyl)amino)-2-((2,3,5,6-tetrafluorophenoxy)methyl)phenoxy)tetrahydro-2H-pyran-3,4,5-triyl triacetate, 13

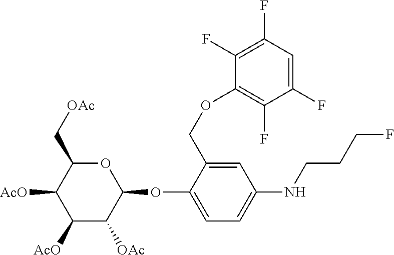 [SMILES: CC(=O)OC[C@H]1O[C@@H](Oc2ccc(NCCCF)cc2COc2c(F)c(F)cc(F)c2F)[C@H](OC(C)=O)[C@@H](OC(C)=O)[C@H]1OC(C)=O]

A solution of 10 (43.0 mg, 0.06 mmol) in DCM/TFA 1:1 v/v (0.85 mL, 0.85 mL) was stirred for 15 min at room temperature. The solvent was evaporated under reduced pressure and the residue dissolved in dry MeCN (1.00 mL) under argon atmosphere. NaI (4.08 mg, 0.03 mmol), Et₃N (0.04 mL, 0.26 mmol) and 3-fluoro-1-iodopropane (0.01 mL, 0.08 mmol) were added. The mixture was heated overnight at 80° C. The reaction mixture was diluted with water and extracted with EtOAc. The organic phase was dried over MgSO₄, evaporated and purified by flash chromatography (PE/EtOAc 16% to 100% B) to afford the product (11.0 mg, 25%).

### Synthesis of Compound 14

### (2S,3R,4S,5R,6R)-2-(4-((3-fluoropropyl)amino)-2-((2,3,5,6-tetrafluorophenoxy)methyl)phenoxy)-6-(hydroxymethyl)tetrahydro-2H-pyran-3,4,5-triol, 14

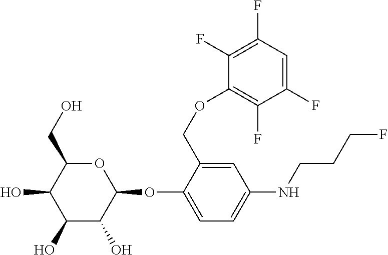 [SMILES: OC[C@H]1O[C@@H](Oc2ccc(NCCCF)cc2COc2c(F)c(F)cc(F)c2F)[C@H](O)[C@@H](O)[C@H]1O]

A solution of 13 (2.00 mg, 3.00 μmol) in MeOH/H₂O/Et₃N 10:1:1 v/v (0.50 mL, 0.05 mL, 0.05 mL) was stirred for 90 min at room temperature. The solvent was evaporated under reduced pressure to afford the product. ¹H NMR (600 MHz, DMSO-d₆) δ 7.57 (q, J=8.9 Hz, 1H), 6.97 (d, J=8.8 Hz, 1H), 6.63 (d, J=2.9 Hz, 1H), 6.52 (dd, J=8.8, 2.9 Hz, 1H), 5.45 (t, J=5.9 Hz, 1H), 5.35 (d, J=11.5 Hz, 1H), 5.29 (d, J=11.6 Hz, 1H), 5.03 (d, J=5.3 Hz, 1H), 4.79 (s, 1H), 4.61 (s, 1H), 4.57 (t, J=5.9 Hz, 1H), 4.49 (t, J=5.9 Hz, 1H), 4.44 (d, J=7.8 Hz, 2H), 3.66 (t, J=3.8 Hz, 2H), 3.28 (d, J=4.8 Hz, 2H), 3.05 (q, J=4.2, 3.2 Hz, 2H), 1.87 (dp, J=25.8, 6.4 Hz, 2H).

### Example 4

Example 4 discloses the precursor synthesis of compounds according to reaction scheme 6.

### Synthesis of Compound 15

### tert-butyl 4-(4-hydroxyphenyl)piperazine-1-carboxylate, 15

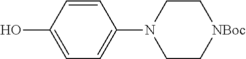 [SMILES: CC(C)(C)OC(=O)N1CCN(c2ccc(O)cc2)CC1]

Boc₂O (1.29 g, 5.9 mmol) in DCM 5 (mL) was added dropwise to an ice-cooled solution of 4-(piperazin-1-yl)phenol (1.0 g, 5.6 mmol) and Et₃N (1.1 mL) in DCM (10 mL) under an argon atmosphere. The reaction mixture was stirred overnight, warming slowly to room temperature. The reaction mixture was washed with 10% aqueous citric acid (3×20 mL), water (2×10 mL) and brine (10 mL), after which it was dried over MgSO₄ and concentrated under reduced pressure. The resulting residue was purified using silica gel flash chromatography (10-100% EtOAc in PE), affording the product as a white solid (1.0 g, 64.3%). R_{f}: 0.50 (PE/EtOAc 1:1); ¹H NMR (600 MHz, CDCl₃) δ 6.94-6.87 (m, 2H), 6.81-6.76 (m, 2H), 3.64-3.55 (m, 4H), 3.02 (t, J=5.1 Hz, 4H), 1.48 (s, 9H).

### Synthesis of Compound 16

### (2R,3S,4S,5R,6S)-2-(acetoxymethyl)-6-(4-(4-(tert-butoxycarbonyl)piperazin-1-yl)phenoxy)tetrahydro-2H-pyran-3,4,5-triyl triacetate, 16

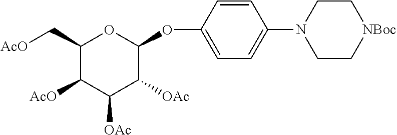 [SMILES: CC(=O)OC[C@H]1O[C@@H](Oc2ccc(N3CCN(C(=O)OC(C)(C)C)CC3)cc2)[C@H](OC(C)=O)[C@@H](OC(C)=O)[C@H]1OC(C)=O]

A solution of acetobromogalactose (3.0 g, 7.3 mmol), compound 15 (3.1 g, 10.8 mmol) and NaOH (430 mg, 10.8 mmol) in 1:1 acetone/water (48 mL) was stirred overnight at room temperature. The reaction mixture was diluted with EtOAc (150 mL), after which it was washed with water (3×30 mL) and brine (30 mL). The organic layer was dried over MgSO₄, concentrated under reduced pressure and the resulting residue purified using silica gel flash chromatography (10-80% EtOAc in PE), affording the product as a colorless oil. R_{f}: 0.45 (PE/EtOAc 1:1); MS (ESI): [M+H]⁺ (theor.)=608.2, Measured=609.2

### Synthesis of Compound 17

### (2R,3S,4S,5R,6S)-2-(acetoxymethyl)-6-(4-(4-(3-hydroxypropyl)piperazin-1-yl)phenoxy)tetrahydro-2H-pyran-3,4,5-triyl triacetate, 17

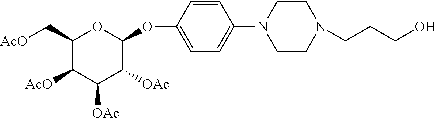 [SMILES: CC(=O)OC[C@H]1O[C@@H](Oc2ccc(N3CCN(CCCO)CC3)cc2)[C@H](OC(C)=O)[C@@H](OC(C)=O)[C@H]1OC(C)=O]

A solution of the N-Boc protected glycoside 16 (385 mg, 0.63 mmol) in 1:1 TFA/DCM (4 mL) was stirred at room temperature for 1 hour. TLC analysis revealed the consumption of the starting material and the appearance of a single polar product. The solvents were removed under high vacuum and the resulting residue dissolved in CH₃CN (10 mL). To this solution was added 3-bromopropan-1-ol (106 mg, 0.76 mmol), Et₃N (335 μL) and a catalytic amount of NaI. The reaction was refluxed under an argon atmosphere for 3 hours, after which the solvent was removed under reduced pressure. The resulting residue was taken up into DCM (20 mL), washed with water (10 mL) and brine (10 mL) and dried over MgSO₄. Purification using silica gel flash chromatography (0-10% MeOH/DCM) afforded the product as a colorless oil (210 mg, 58.7%). Rr: 0.45 (MeOH/DCM 1:9); HPLC single peak. MS (ESI): [M+H]⁺ (theor.)=567.2, Measured=567.2

### Synthesis of Compound 18

### (2R,3S,4S,5R,6S)-2-(acetoxymethyl)-6-(4-(4-(3-((methylsulfonyl)oxy)propyl)piperazin-1-yl)phenoxy)tetrahydro-2H-pyran-3,4,5-triyl triacetate, 18

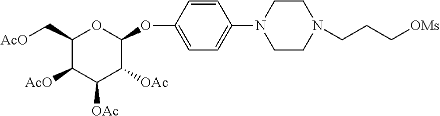 [SMILES: CC(=O)OC[C@H]1O[C@@H](Oc2ccc(N3CCN(CCCOS(C)(=O)=O)CC3)cc2)[C@H](OC(C)=O)[C@@H](OC(C)=O)[C@H]1OC(C)=O]

MsCl (43 μL, 0.56 mmol) in DCM (1 mL) was added dropwise to an ice-cooled solution of alcohol 17 (210 mg, 0.37 mmol) and Et₃N (234 μL) in DCM (4 mL), under an argon atmosphere. The reaction was allowed to warm to room temperature, after which saturated aqueous NaHCO₃ (10 mL) was added. The mixture was extracted into DCM (3×10 mL), washed with water (10 mL) and brine (10 mL) and dried over Na₂SO₄. After concentrating under reduced pressure, the residue was purified using silica gel flash chromatography (0-15% MeOH/DCM), affording the product as a colorless oil (200 mg, 83.8%). R_{f}: 0.5 (MeOH/DCM 1:9); HPLC single peak. MS (ESI): [M+H]⁺ (theor.)=645.7, Measured=645.2

### Synthesis of Compound 19

### (2R,3S,4S,5R,6S)-2-(acetoxymethyl)-6-(4-(4-(3-fluoropropyl)piperazin-1-yl)phenoxy)tetrahydro-2H-pyran-3,4,5-triyl triacetate, 19

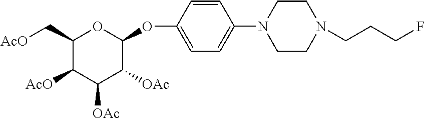 [SMILES: CC(=O)OC[C@H]1O[C@@H](Oc2ccc(N3CCN(CCCF)CC3)cc2)[C@H](OC(C)=O)[C@@H](OC(C)=O)[C@H]1OC(C)=O]

A solution of the N-Boc protected glycoside 16 (401 mg, 0.66 mmol) in 1:1 TFA/DCM (4 mL) was stirred at room temperature for 1 hour. TLC analysis revealed the consumption of the starting material and the appearance of a single polar product. The solvents were removed under high vacuum and the resulting residue dissolved in CH₃CN (10 mL). To this solution was added 3-fluoro-1-iodopropane (138 mg, 0.74 mmol) and Et₃N (930 μL). The reaction was heated to 50° C. overnight under an argon atmosphere. The solvent was removed under reduced pressure. The resulting residue was taken up into DCM (20 mL), washed with water (10 mL) and brine (10 mL) and dried over MgSO₄. Purification using silica gel flash chromatography (0-10% MeOH/DCM) afforded the product as a colorless oil (31 mg, 8.3%). R_{f}: 0.45 (MeOH/DCM 1:9); HPLC single peak. MS (ESI): [M+H]⁺ (theor.)=569.3, Measured=569.3; ¹H NMR (600 MHz, DMSO) δ 7.02-6.97 (m, 2H), 6.97-6.86 (m, 2H), 5.34-5.26 (m, 3H), 5.18 (dd, J=10.3, 7.9 Hz, 1H), 4.61 (t, J=5.6 Hz, 1H), 4.53 (t, J=5.7 Hz, 1H), 4.39 (ddd, J=7.0, 5.6, 1.2 Hz, 1H), 4.15-4.06 (m, 2H), 3.73 (s, 1H), 3.62 (s, 1H), 3.19 (s, 2H), 3.12 (qd, J=7.3, 4.6 Hz, 2H), 2.98 (d, J=15.5 Hz, 2H), 2.15 (s, 3H), 2.05 (s, 2H), 2.02 (s, 2H), 1.95 (s, 2H), 1.20 (t, J=7.3 Hz, 4H).

### Synthesis of Compound 20

### (2S,3R,4S,5R,6R)-2-(4-(4-(3-fluoropropyl)piperazin-1-yl)phenoxy)-6-(hydroxymethyl)tetrahydro-2H-pyran-3,4,5-triol, 20

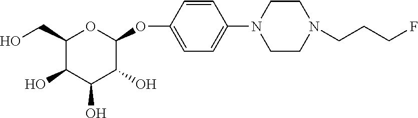 [SMILES: OC[C@H]1O[C@@H](Oc2ccc(N3CCN(CCCF)CC3)cc2)[C@H](O)[C@@H](O)[C@H]1O]

The protected glycoside 19 (50 mg, 0.09 mmol) was stirred in a 1:1:8 mixture of Et₃N/H₂O/MeOH at room temperature for 2 hours. The residue was purified using preparative HPLC, which after lyophilization afforded the cold standard (28 mg, 78%). HPLC single peak. MS (ESI): [M+H]⁺ (theor.)=401.2, Measured=401.3 Example 5

Example 5 discloses the precursor synthesis of compounds according to reaction schemes 7 and 8.

### Synthesis of Compound 21

### (2R,3S,4S,5R,6S)-2-(acetoxymethyl)-6-(2-formyl-5-iodophenoxy)tetrahydro-2H-pyran-3,4,5-triyl triacetate, 21

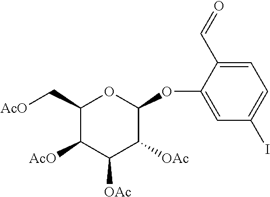 [SMILES: CC(=O)OC[C@H]1O[C@@H](Oc2cc(I)ccc2C=O)[C@H](OC(C)=O)[C@@H](OC(C)=O)[C@H]1OC(C)=O]

Diisopropylethyl amine (1.1 mL) was added dropwise to an ice cooled solution of acetobromogalactose (1.0 g, 2.4 mmol) and 5-iodo-salicylaldehyde (0.5 g, 2.0 mmol) in CH₃CN (15 mL), under an argon atmosphere. The mixture was left to stir over night while warming slowly to room temperature. After concentrating the mixture under reduced pressure, resulting residue was diluted with EtOAc (100 mL), after which it was washed successively with 5% citric acid (3×50 mL), water (2×50 mL), saturated aqueous NaHCO₃ (2×50 mL) and brine (50 mL). The organic layer was then dried over Na₂SO₄, after it was concentrated under reduced pressure. The mixture purified using silica gel flash chromatography (10-100% EtOAc/PE) to afford the product as a colorless foam (1.0 g, 85.6%). ¹H NMR (600 MHz, CDCl₃) δ 10.22 (s, 1H), 8.13 (d, J=2.3 Hz, 1H), 7.82 (dd, J=8.7, 2.3 Hz, 1H), 6.90 (d, J=8.7 Hz, 1H), 5.56 (dd, J=10.5, 7.9 Hz, 1H), 5.47 (dd, J=3.4, 1.1 Hz, 1H), 5.16-5.09 (m, 2H), 4.25-4.07 (m, 3H), 2.19 (s, 3H), 2.06 (s, 3H) 2.05 (s, 3H), 2.02 (s, 3H).

### Synthesis of Compound 22

### (2R,3S,4S,5R,6S)-2-(acetoxymethyl)-6-(2-(hydroxymethyl)-5-iodophenoxy)tetrahydro-2H-pyran-3,4,5-triyl triacetate, 22

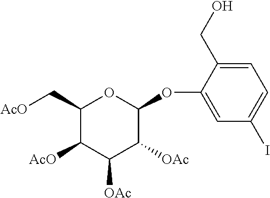 [SMILES: CC(=O)OC[C@H]1O[C@@H](Oc2cc(I)ccc2CO)[C@H](OC(C)=O)[C@@H](OC(C)=O)[C@H]1OC(C)=O]

A solution of the aldehyde 21 (0.8 g, 1.4 mmol) in DCM (18 mL) and isopropanol (6 mL) was prepared. To this was added sodium borohydride (0.2 g, 5.3 mmol) slowly in small portions. The reaction was allowed to stir at room temperature for 1 hour, after which 5% Citric acid (10 mL) was added. The mixture was extracted into DCM (3×20 mL) and the combined organics were washed with saturated aqueous NaHCO₃ (2×15 mL), water (15 mL) and brine (10 mL), before being dried over MgSO₄ and concentrated under reduced pressure. Purification using silica gel flash chromatography (10-100% EtOAc/PE) afforded the product (430 mg, 54%). HPLC single peak. MS (ESI): [M+Na]+(theor.)=603.0, Measured=603.0

### Synthesis of Compound 23

### (2R,3S,4S,5R,6S)-2-(acetoxymethyl)-6-(5-iodo-2-(((methylsulfonyl)oxy)methyl)phenoxy)tetrahydro-2H-pyran-3,4,5-triyl triacetate, 23

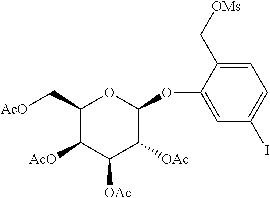 [SMILES: CC(=O)OC[C@H]1O[C@@H](Oc2cc(I)ccc2COS(C)(=O)=O)[C@H](OC(C)=O)[C@@H](OC(C)=O)[C@H]1OC(C)=O]

A solution of the alcohol 22 (660 mg, 1.1 mmol) in dry DCM (10.0 mL) under argon atmosphere was cooled down to 0° C. and Et₃N (0.5 mL) was added. MsCl (156 mg, 1.4 mmol) in DCM (1 mL) was added dropwise and the mixture was stirred for 1 hour at 0° C. The reaction mixture was diluted with water and extracted with DCM (3×10 mL). The combined organic phases were dried over MgSO₄, evaporated and purified by flash chromatography (10-100% PE/EtOAc) to afford the mesylate product (540 mg, 72.6%). HPLC single peak. MS (ESI): [M+Na]⁺ (theor.)=681.4, Measured=681.0

### Synthesis of Compound 24

### (2R,3S,4S,5R,6S)-2-(acetoxymethyl)-6-(5-iodo-2-((2,3,5,6-tetrafluorophenoxy)methyl)phenoxy)tetrahydro-2H-pyran-3,4,5-triyl triacetate, 24

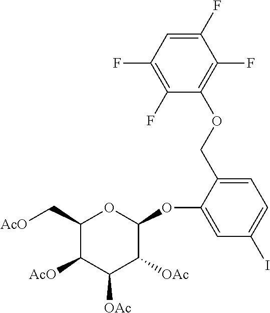 [SMILES: CC(=O)OC[C@H]1O[C@@H](Oc2cc(I)ccc2COc2c(F)c(F)cc(F)c2F)[C@H](OC(C)=O)[C@@H](OC(C)=O)[C@H]1OC(C)=O]

The mesylate intermediate 23 (202 mg, 0.3 mmol), Cs₂CO₃ and 2,3,5,6-tetrafluorophenol were added to CH₃CN (10 mL). The mixture was stirred overnight at room temperature. The reaction mixture was diluted with water and extracted with EtOAc to afford the product (160 mg, 71.7%). HPLC single peak. (ESI): [M+Na]⁺ (theor.)=751.0, Measured=750.9

### Synthesis of Compound 25

### (2R,3S,4S,5R,6S)-2-(acetoxymethyl)-6-(2-((2,3,5,6-tetrafluorophenoxy)methyl)-5-(4,4,5,5-tetramethyl-1,3,2-dioxaborolan-2-yl)phenoxy)tetrahydro-2H-pyran-3,4,5-triyl triacetate, 25

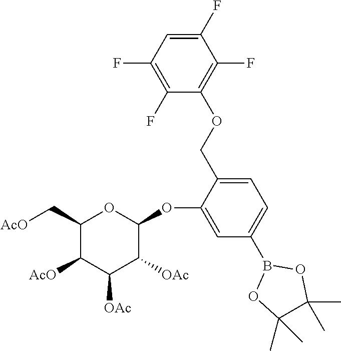 [SMILES: CC(=O)OC[C@H]1O[C@@H](Oc2cc(B3OC(C)(C)C(C)(C)O3)ccc2COc2c(F)c(F)cc(F)c2F)[C@H](OC(C)=O)[C@@H](OC(C)=O)[C@H]1OC(C)=O]

A solution containing the iodide precursor 24 (160 mg, 0.2 mmol), potassium acetate (21.6 mg, 0.7 mmol), Pd(dppf)Cl₂ (33 mg, 44 μmol), B₂pin₂ (67 mg, 0.26 mmol) in DMF (5 mL) was stirred under an argon atmosphere at 80° C. overnight. The reaction mixture was diluted with water (50 mL) and extracted into DCM (3×10 mL). The combined organic layers were washed with water (10 mL) and brine (10 mL), before being dried over MgSO₄. Purified by silica gel flash chromatography (10-100% PE/EtOAc) afforded the product as a colorless oil (80 mg, 55.0%). ¹H NMR (600 MHz, CDCl₃) δ 7.79 (s, 1H), 7.75 (d, J=8.2 Hz, 1H), 7.03 (d, J=8.2 Hz, 1H), 6.75 (tt, J=9.9, 6.9 Hz, 1H), 5.49 (dd, J=10.5, 7.9 Hz, 1H), 5.46 (d, J=3.4 Hz, 1H), 5.37 (d, J=11.5 Hz, 1H), 5.16-5.10 (m, 2H), 5.07 (d, J=11.5 Hz, 1H), 4.22-4.07 (m, 4H), 2.19 (s, 3H), 2.07 (s, 3H), 2.00 (s, 3H), 1.97 (s, 3H), 1.32 (s, 12H).

### Synthesis of Compound 26

### (2R,3S,4S,5R,6S)-2-(acetoxymethyl)-6-(5-fluoro-2-formylphenoxy)tetrahydro-2H-pyran-3,4,5-triyl triacetate, 26

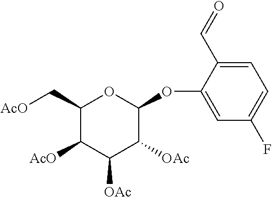 [SMILES: CC(=O)OC[C@H]1O[C@@H](Oc2cc(F)ccc2C=O)[C@H](OC(C)=O)[C@@H](OC(C)=O)[C@H]1OC(C)=O]

Diisopropylethyl amine (930 μL) was added dropwise to an ice cooled solution of acetobromogalactose (730 mg, 1.8 mmol) and 5-fluoro-salicaldehyde (820 mg, 5.8 mmol) in CH₃CN (10 mL), under an argon atmosphere. The mixture was left to stir overnight while warming slowly to room temperature. After concentrating the mixture under reduced pressure, resulting residue was diluted with EtOAc (500 mL), after which it was washed successively with 5% citric acid (3×25 mL), water (2×25 mL), saturated aqueous NaHCO₃ (2×25 mL) and brine (25 mL). The organic layer was then dried over Na₂SO₄, after which it was concentrated under reduced pressure. The mixture purified using silica gel flash chromatography to afford the product (505 mg, 59.4%). (ESI): [M+Na]⁺ (theor.)=493.1, Measured=493.1

### Synthesis of Compound 27

### (2R,3S,4S,5R,6S)-2-(acetoxymethyl)-6-(5-fluoro-2-(hydroxymethyl)phenoxy)tetrahydro-2H-pyran-3,4,5-triyl triacetate, 27

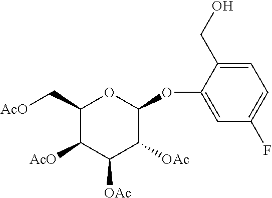 [SMILES: CC(=O)OC[C@H]1O[C@@H](Oc2cc(F)ccc2CO)[C@H](OC(C)=O)[C@@H](OC(C)=O)[C@H]1OC(C)=O]

The aldehyde 26 (200 mg, 430 μmol) was dissolved in a mixture DCM (9 mL) and isopropanol (3 mL). NaBH₄ (135 mg, 3.6 mmol) was added slowly and the reaction was stirred at room temperature for 2 hours. To quench the reaction, 5% Citric acid (10 mL) was added. The mixture was extracted into DCM (3×20 mL) and the combined organics were washed with saturated aqueous NaHCO₃ (2×15 mL), water (15 mL) and brine (10 mL), before being dried over MgSO₄ and concentrated under reduced pressure. Purification using silica gel flash chromatography (10-100% EtOAc/PE) afforded the product (110 mg, 55.0%). (ESI): [M+Na]⁺ (theor.)=495.1, Measured=495.2

### Synthesis of Compound 28

### (2R,3S,4S,5R,6S)-2-(acetoxymethyl)-6-(5-fluoro-2-((2,3,5,6-tetrafluorophenoxy)methyl)phenoxy)tetrahydro-2H-pyran-3,4,5-triyl triacetate, 28

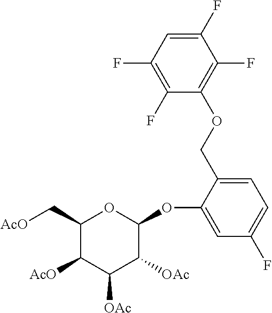 [SMILES: CC(=O)OC[C@H]1O[C@@H](Oc2cc(F)ccc2COc2c(F)c(F)cc(F)c2F)[C@H](OC(C)=O)[C@@H](OC(C)=O)[C@H]1OC(C)=O]

A solution of the alcohol 27 (200 mg, 360 μmol) in dry DCM (10.0 mL) under argon atmosphere was cooled down to 0° C. and Et₃N (0.5 mL) was added. MsCl (100 mg, 872 μmol) in DCM (1 mL) was added dropwise and the mixture was stirred for 1 hour at 0° C. The reaction mixture was diluted with water and extracted with DCM (3×10 mL). The combined organic phases were dried over MgSO₄, evaporated to afford the mesylate intermediate (115 mg, 58.1%).

The mesylate intermediate (115 mg, 209 μmol), Cs₂CO₃ (136 mg, 418 μmol) and 2,3,5,6-tetrafluorophenol (52.5 mg, 314 μmol) were added to CH₃CN (10 mL). The mixture was stirred overnight at room temperature. The reaction mixture was diluted with water and extracted with EtOAc to afford the product (92 mg, 41.1%). HPLC single peak. (ESI): [M+Na]⁺ (theor.)=643.1, Measured=643.1

### Synthesis of Compound 29

### (2S,3R,4S,5R,6R)-2-(5-fluoro-2-((2,3,5,6-tetrafluorophenoxy)methyl)phenoxy)-6-(hydroxymethyl)tetrahydro-2H-pyran-3,4,5-triol, 29

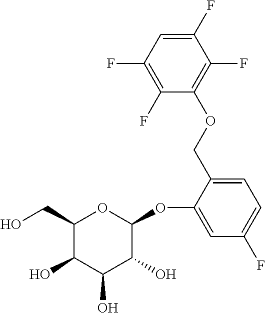 [SMILES: OC[C@H]1O[C@@H](Oc2cc(F)ccc2COc2c(F)c(F)cc(F)c2F)[C@H](O)[C@@H](O)[C@H]1O]

The acetylated intermediate 28 (65 mg) was stirred for 2 hours at room temperature in MeOH (15 mL) with a catalytic amount of NaOMe. Amberlite IR120 (1 g) was added and the solution was filtered and concentrated under reduced pressure to afford the cold standard (quant). HPLC single peak. MS (ESI): [M+H]⁺ (theor.)=453.1, Measured=453.0

### Example 6

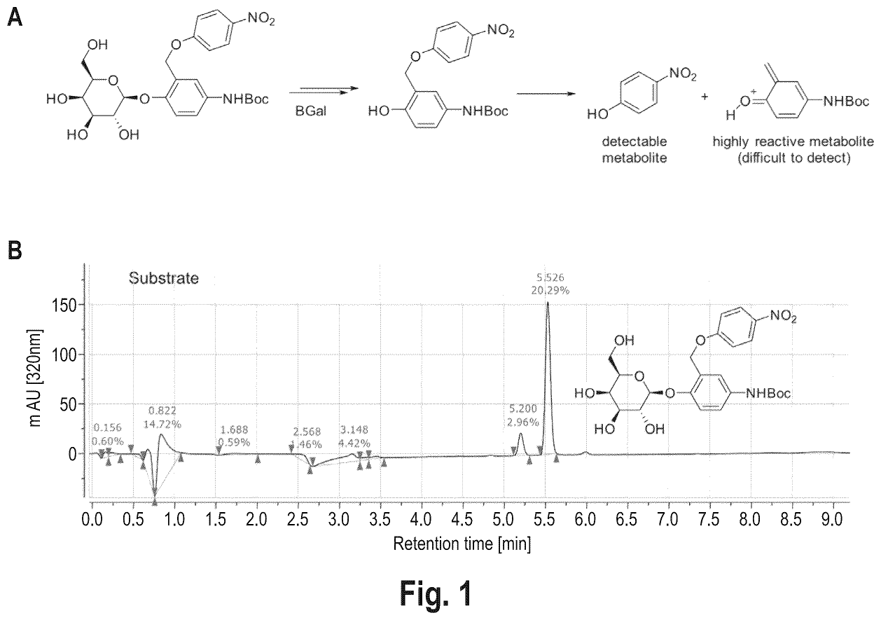

With reference to FIG. 1, example 6 discloses a model experiment with a cold tracer to validate the self-immolation mechanism.

The model substrate, shown at the left of FIG. 1A, was incubated with beta-galactosidase. Enzyme hydrolysis of the substrate generates the primary metabolite, which spontaneously decomposed into the quinone methide and an easily detectable colored metabolite (4-nitrophenol). In particular, FIG. 1A shows schematic of enzymatic reaction; FIG. 1B: HPLC of the intact substrate; FIG. 1C: HPLC of enzyme and substrate showing the easily detectable para-nitrophenol (4NP) metabolite after 30 min. incubation; FIG. 1D: HPLC of reference 4NP. 4NP is detectable by absorption measurement at a wavelength of 320 nm.

### Example 7

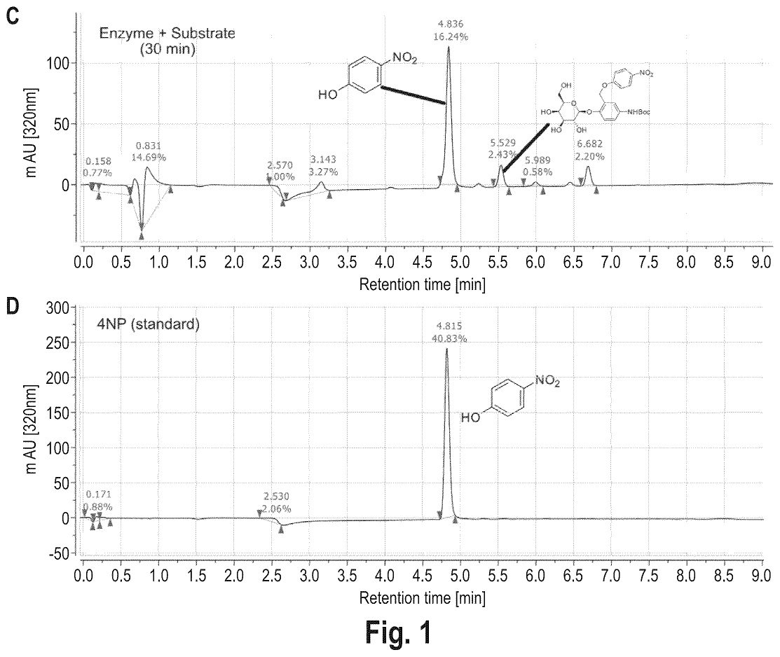

An uptake experiment in beta galactosidase (BG) over-expressing cells is shown in FIG. 2.

In vitro experiments were performed by uptake quantification in a gamma-counter. In all experiments, 1 million of each of the cell lines were treated with 20 μCi of tracer. After incubation time of 40 minutes, the cells were centrifuged, the supernatant discarded and the cell pellet resuspended in fresh PBS. The samples were then measured in biodistribution tubes in a gamma counter. The tracers were tested first using a β-galactosidase (LacZ) overexpression model of a colon carcinoma cell line (CT26-CL25) and the wild type (CT26) as control (FIG. 2A). Next, the tracers were tested in a senescent cell model: a liver carcinoma cell line (Amp19) treated with ribosomal checkpoint inhibitor (RCI) (FIGS. 2B and 2C).

### Example 8

Example 8 shows uptake experiments in senescence cell models.

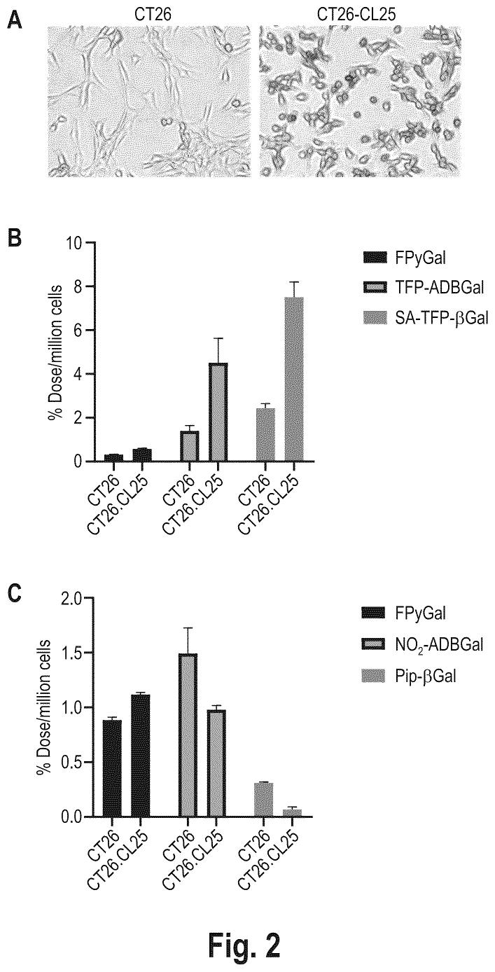

With reference to FIGS. 3A and 3B, results of in vitro evaluation of new tracers in senescence models are summarized in table 1 below.

TABLE 1

| | Uptake in % ID per million cells | | | | | |
|---|---|---|---|---|---|---|
| | | CT26.CL25 | Amp19 | Amp19 | HCT116 | HCT116 |
| Tracer | CT26 | (LacZ+) | (sen) | (cntrl) | (sen) | (cntrl) |
| [¹⁸F]FPyGal | 0.31788587 | 0.5769262 | 0.06390066 | 0.26566235 | 0.1998162 | 0.34376656 |
| [¹⁸F]ADTFPBGal | 1.40338331 | 4.51479494 | 0.20498739 | 1.08458394 | 0.6573454 | 1.56425247 |
| [¹⁸F]TFPBGal | 2.44369202 | 7.51394126 | 0.39922051 | 3.6413569 | 2.7607024 | 4.74515548 |
| [¹⁸F]FPyGal | 0.884193063 | 1.116845733 | — | — | — | — |
| [¹⁸F]NO₂BGal | 1.491827057 | 0.979156165 | 0.32451660 | 1.28576145 | 0.3025945 | 0.50524818 |
| [¹⁸F]PIPGal | 0.3131021 | 0.068978193 | 0.04018398 | 0.31653522 | 0.0984338 | 0.12194638 |

In course of the in vivo experiments, senescence was induced in vivo in Amp19 mouse xenograft by treatment with RCl. For the control group, the mice were treated with vehicle (PBS). After inducing senescence, the tracer (13 MBq) was injected i.v. and dynamic PET and MRI scans were performed. Ex vivo tumors sections were used for autoradiography, X-Gal staining and immunohistochemistry (H&E, p53, p21 and ki-67).

### Example 9

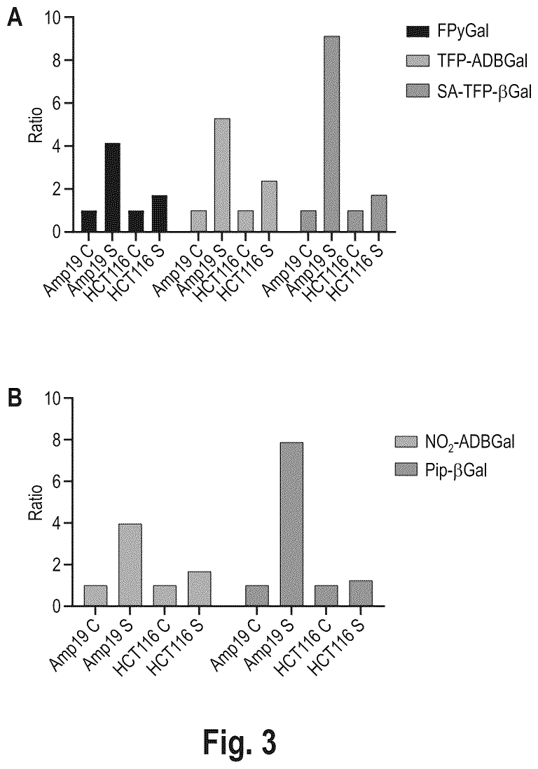

Dynamic PET scans of Amp19 tumor bearing mice with [¹⁸F]TFPBGal have been performed in example 9 (FIG. 4). The dynamic uptake of [¹⁸F]TFPBGal is higher in CX-treated (senescent) tumors compared to untreated controls at all time points. In both treated and untreated tumors, the uptake is stable and reaches a maximum and plateaus, with no observable washout effect.

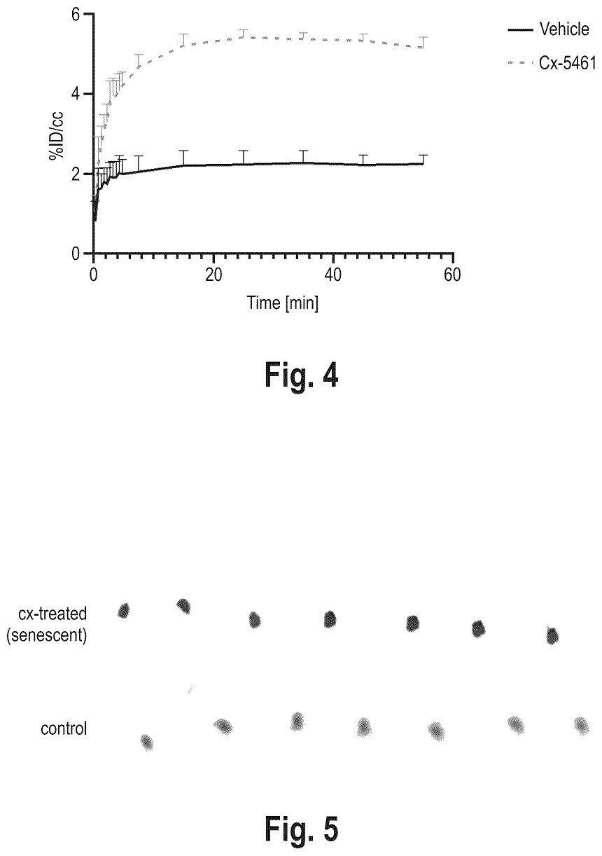

With reference to FIG. 5, an autoradiography of cx-treated (senescent) and control Amp19 tumors after administration of [¹⁸F]PIPGal is shown. Autoradiography was performed after administering [¹⁸F]PIPGal to mice bearing Amp19 tumors. High signal accumulation is observed in the cx-treated (senescent) tumors compared to the non-senescent controls.

The autoradiography of the tumors (FIG. 5) shows an increase of the uptake in in CX-treated (senescent) tumors compared to untreated controls after the PET scan.

The tumors were further analyzed with conventional H&E staining. Additionally, Ki67, activated Caspase 3, p53, p21 and CD31 IHC were performed in all samples.

The pathology report indicated that the control tumors showed more necrosis, higher proliferation and apoptosis rates, less p21 and p53 positive cells, and more blood vessels compared to the senescent tumors.

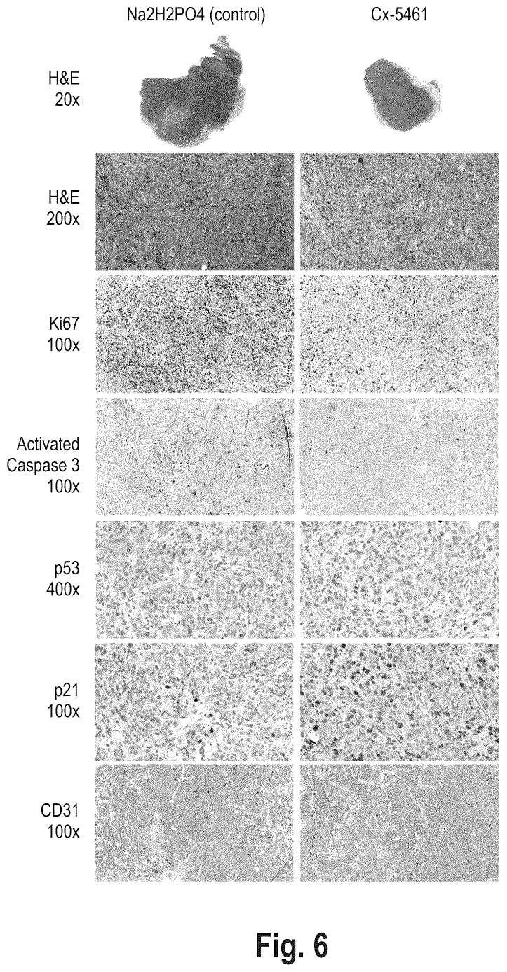

Ex vivo immunohistochemistry of cx-treated (senescent) and vehicle-treated (control) Amp19 tumors is shown in FIG. 6 and table 2.

TABLE 2

| | | | | | Ki67 | | | | | |
|---|---|---|---|---|---|---|---|---|---|---|
| | | | | | (% of | | | | | |
| sample | Tumor | inflammation | inflammation | | positive | activated | | | | |
| ID | size | periphery | central | necrosis | cells) | caspase 3 | p53 | p21 | CD31 | comments |
| 22A23.1 | small | mild to | low | +++ | 50-70% | ++ | <5% | 5-10% | ++ | |
| | | moderate | | | | | | | | |
| 22A23.2 | small | low | low | − | 20-30% | + | 15-25% | 20-30% | ++ | increased |
| | | | | | | | | | | firbrosis |
| 22A23.3 | small | moderate | low | ++ | 60-80% | + | <5% | 1-2% | ++/+++ | |
| 22A23.4 | small | low | low | + | 20-30% | + | 15-25% | 30-50% | ++ | increased |
| | | | | | | | | | | firbrosis |
| 22A23.5 | small | mild to | low | ++ | 60-80% | ++ | <5% | 5% | +++ | |
| | | moderate | | | | | | | | |
| 22A23.6 | small | low | low | − | 20-30% | + | 30-40% | 30-50% | ++ | increased |
| | | | | | | | | | | firbrosis |

Further, the disclosure comprises examples according to the following clauses:

Clause 1. A compound of the formula:

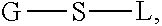

or a stereoisomer, enantiomer, tautomer, prodrug and/or a pharmaceutically acceptable salt thereof,
wherein
G is a glycoside, and a substituted or unsubstituted C₁-C₅ alkyl derivative thereof and/or a substituted or unsubstituted N-acetyl derivative thereof, having a glycosidic bond to S, wherein * represents the binding site between G and S,
S is selected from the group consisting of

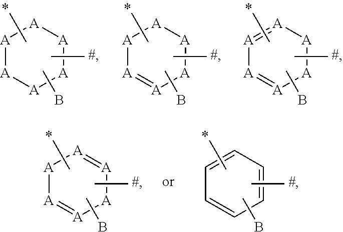

A is independently selected from the group consisting of C, S, N, and O, with the proviso that at least 3 C-atoms are present;
#represents the binding site between S and L;
L is selected from the group consisting of

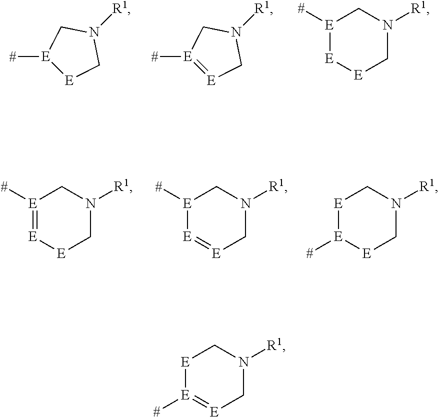

#—CH₂OR², or #—CH₂OOCNHR²;
E is independently selected from the group consisting of N, NH, C, CH, and CH₂;
B and R¹ are independently selected from the group consisting of R³Z, H, F, Cl, Br, I, NO₂, OR⁴, NR⁴R⁵, and substituted or unsubstituted C₁-C₅ alkyl, provided that at least one R³Z is present;
R² is selected from the group consisting of substituted or unsubstituted alkyl, substituted or unsubstituted alkenyl, substituted or unsubstituted aryl, and substituted or unsubstituted heteroaryl;
R³Z is selected from the group consisting of (CH₂)ₘZ, [(CH₂)ₘO(CH₂)ₙ]ₒZ, [(CH₂)ₘCO(CH₂)ₙ]ₒZ, [(CH₂)ₘOOC(CH₂)ₙ]ₒZ, [(CH₂)ₘNR⁴(CH₂)ₙ]ₒZ, wherein m is 0 to 5, and n and o are independently from each other integers from 1 to 5;
R⁴ and R⁵ are independently selected from the group consisting of H, and substituted or unsubstituted C₁-C₅ alkyl; and
Z is a radioactive detectable label, a radioactive therapeutic residue, a chelator coordinating a radioactive detectable label or a chelator coordinating a radioactive therapeutic residue.

Clause 2. The compound according to clause 1, wherein
the substituted or unsubstituted C₁-C₅ alkyl derivative of the glcyoside is a substituted or unsubstituted 5-(C₁-C₅ alkyl)glycoside; and/or
the substituted or unsubstituted N-acetyl derivative of the glycoside is a substituted or unsubstituted 2-(N-acetyl)glycoside, a substituted or unsubstituted 3-(N-acetyl)glycoside, or a substituted or unsubstituted poly(N-acetyl)glycoside; and/or
the glycoside is selected from the group consisting of α-D-glucofuranoside, α-D-mannofuranoside, α-D-fructofuranoside, α-D-glucopyranoside, α-D-mannopyranoside, α-D-galactopyranoside, α-D-fructopyranoside, β-D-glucofuranoside, β-D-mannofuranoside, β-D-fructofuranoside, β-D-glucopyranoside, β-D-mannopyranoside, β-D-galactopyranoside, and β-D-fructopyranoside; and/or
R³Z is selected from the group consisting of Z, [(CH₂)ₘO(CH₂)ₙ]ₒZ, [(CH₂)ₘCO(CH₂)ₙ]ₒZ, NR⁴(CH₂)ₙZ, wherein R⁴ is H, methyl or ethyl, wherein m, n and o are independently from each other integers from 1 to 5; and/or

Clause 3. The compound according to any of the preceding clauses, wherein
L is

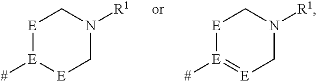

preferably

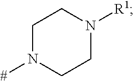

R¹ is independently selected from the group consisting of (CH₂)ₙZ, or O(CH₂)ₙZ, wherein n is 1 to 3, preferably wherein #is in meta-position or para-position to *;
B is independently selected from the group consisting of H, F, Cl, Br, I, NO₂, OR⁴, NR⁴R⁵, and unsubstituted C₁-C₅ alkyl, provided that at least three H are present, wherein R⁴ and R⁵ are independently selected from the group consisting of H, methyl, or ethyl.

Clause 4. The compound according to any of clauses 1 to 3, wherein
L is #—CH₂OR² or #—CH₂OOCNHR², preferably wherein L is in ortho-position or para-position to *;
R² is substituted or unsubstituted C₁ to C₈ alkyl, substituted or unsubstituted aryl, preferably substituted benzyl;
B is independently selected from the group consisting of R³Z, H, F, Cl, Br, I, NO₂, and substituted or unsubstituted C₁-C₅ alkyl, provided that at least one R³Z at least two H are present are present;
R³Z is selected from the group consisting of —Z, —[(CH₂)ₘO(CH₂)ₙ]ₒZ, —[(CH₂)ₘCO(CH₂)ₙ]ₒZ, —NH(CH₂)ₙZ, wherein m, n and o are independently from each other integers from 1 to 4, preferably wherein R³Z is in para-position to * or #; and/or
R⁴ and R⁵ are independently selected from the group consisting of H and substituted or unsubstituted C₁ to C₃ alkyl.

Clause 5. The compound according to any of the preceding clauses wherein the glycoside is β-D-glucopyranoside.

Clause 6. The compound according to any of clauses 1 to 4 wherein the glycoside is β-D-mannopyranoside.

Clause 7. The compound according to any of clauses 1 to 4 wherein the glycoside is β-D-galactopyranoside.

Clause 8. The compound according to any of clauses 1 to 4 wherein the glycoside is β-D-fructopyranoside.

Clause 9. The compound according to any of the preceding clauses wherein S is

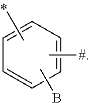

Clause 10. The compound according to any of clauses 1 to 3, and 5 to 9 wherein L is

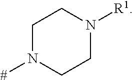

Clause 11. The compound according to clause 10 wherein R¹ is R³Z.

Clause 12. The compound according to any of the preceding clauses wherein B is selected from the group consisting of H, F, Cl, Br, I, NO₂, methyl and ethyl.

Clause 13. The compound according to clause 12 wherein all B with exception of one B are H.

Clause 14. The compound according to clause 13 wherein the one B is selected from the group consisting of F, Cl, Br, I, and methyl.

Clause 15. The compound according to clause 12 wherein all B are H.

Clause 16. The compound according to any of clauses 1 to 3, and 5 to 15 wherein R¹ is (CH₂)ₘZ, wherein m is an integer from 1 to 5.

Clause 17. The compound according to clause 16 wherein m is 2, 3 or 4.

Clause 18. The compound according to clause 16 wherein m is 3.

Clause 19. The compound according to any of clauses 1 to 3, and 5 to 15 wherein R¹ is [(CH₂)ₘO(CH₂)ₙ]ₒZ, wherein m is 0 to 5, and n and o are independently from each other integers from 1 to 5.

Clause 20. The compound according to any of clauses 1 to 3, and 5 to 15 wherein R¹ is [(CH₂)ₘCO(CH₂)ₙ]ₒZ, wherein m is 0 to 5, and n and o are independently from each other integers from 1 to 5.

Clause 21. The compound according to any of clauses 1 to 3, and 5 to 15 wherein R¹ is [(CH₂)ₘOOC(CH₂)ₙ]ₒZ, wherein m is 0 to 5, and n and o are independently from each other integers from 1 to 5.

Clause 22. The compound according to any of clauses 1 to 3, and 5 to 15 wherein R¹ is [(CH₂)ₘNR⁴(CH₂)ₙ]ₒZ, wherein m is 0 to 5, and n and o are independently from each other integers from 1 to 5, wherein R⁴ is H or methyl.

Clause 23. The compound according to any of clauses 19 to 22, wherein m, and o are each 1, and n is 1, 2, or 3.

Clause 24. The compound according to any of clauses 19 to 22, wherein m, n and o are each 1.

Clause 25. The compound according to any of clauses 1 to 2, and 4 to 10 wherein B is selected from the group consisting of R³Z, H, F, Cl, Br, I, NO₂, methyl and ethyl, provided that at least one R³Z is present.

Clause 26. The compound according to clause 25 wherein one B is different from H, the one B being R³Z.

Clause 27. The compound according to any of clauses 25 to 26 wherein R³Z is —Z.

Clause 28. The compound according to any of clauses 25 to 26 wherein R³Z is NH(CH₂)ₙZ, wherein n is an integer from 1 to 4.

Clause 29. The compound according to clause 28, wherein n is 2, 3, or 4.

Clause 30. The compound according to any of clauses 1 to 2, 4 to 10, and 25 to 29 wherein R³Z is in para-position to *.

Clause 31. The compound according to any of clauses 1 to 2, 4 to 10, and 25 to 30, wherein L is #—CH₂OR².

Clause 32. The compound according to any of clauses 1 to 2, 4 to 10, and 25 to 30, wherein L is #—CH₂OOCNHR².

Clause 33. The compound according to any of clauses 1 to 2, 4 to 10, and 25 to 32, wherein R² is aryl or heteroaryl.

Clause 34. The compound according to clause 33, wherein the aryl or the heteroaryl is substituted with at least one residue having a -M effect equal or stronger than NO₂⁻, and/or at least one residue having a —I effect equal or stronger than I⁻.

Clause 35. The compound according to any of clauses 33 to 34, wherein the aryl is substituted with at least one NO₂⁻ and/or F⁻.

Clause 36. The compound according to any of clauses 33 to 35, wherein the aryl is phenyl.

Clause 37. The compound according to any of clauses 33 to 36, wherein the aryl comprises one or two residues having the -M effect, wherein said residues having the -M effect are located in ortho or para position to L.

Clause 38. The compound according to any of clauses 33 to 36, wherein the aryl comprises two or four residues having the —I effect, wherein said residues having the —I effect are located in ortho or meta position to L.

Clause 39. The compound according to any of clauses 33 to 36, wherein the aryl comprises two residues having the —I effect located in ortho position to L and one residue having the -M effect located in para position to L.

Clause 40. The compound according to any of clauses 33 to 35 and 37 to 39, wherein the aryl is naphthyl.

Clause 41. The compound according to clause 1 or 2, wherein the compound is

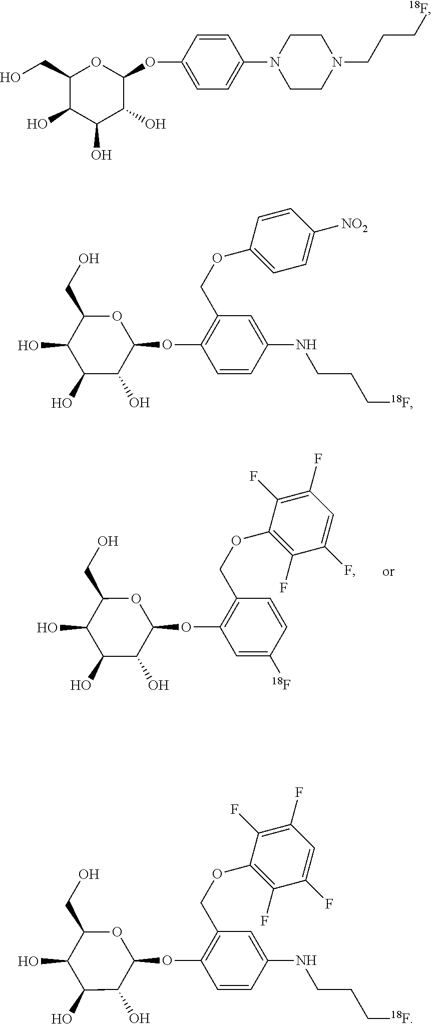

Clause 42. The compound according to any of the preceding clauses adapted to accumulate intracellularly.

Clause 43. The compound according to clause 42, wherein accumulating intracellularly includes bonding of the compound.

Clause 44. The compound according to any of the preceding clauses, wherein the radioactive detectable label is selected from the group consisting of ¹¹C, ⁴⁰K, ¹³N, ¹⁵O, ¹⁸F, ⁷⁵Br, ⁷⁶Br, ⁸²Rb, ⁶⁸Ga, ⁶⁴Cu, ⁶²Cu, ⁸⁹Zr, ¹²³I, ¹²⁴I, ¹²⁵I, ¹³¹I, ²¹⁰At, ²¹¹At and ¹¹¹In.

Clause 45. The compound according to any of the preceding clauses, wherein the radioactive detectable label is selected from the group consisting of ¹¹C, ¹⁸F, ⁶⁸Ga, ⁶⁴Cu, and ¹²⁴I.

Clause 46. The compound according to any of the preceding clauses, wherein the radioactive detectable label is ¹⁸F.

Clause 47. The compound according to any of clauses 1 to 39, wherein the radioactive therapeutic residue is selected from the group consisting of ³²P, ⁶⁰Co, ⁶⁴Cu, ⁸⁹Sr, ⁹⁰Y ¹⁷⁷Lu, ¹⁸⁶Re and ¹⁵³Sm.

Clause 48. The compound according to any of clauses 1 to 40, wherein the radioactive therapeutic residue is ⁶⁴Cu.

Clause 49. The compound according to any of the preceding clauses for use in surgery.

Clause 50. The compound according to any of clauses 1 to 46 for use in a method for detecting cell senescence.

Clause 51. The compound according to any one of clauses 1 to 46 for use in a method of determining the efficiency of cancer treatment.

Clause 52. Method for detecting cell senescence comprising contacting cells with a compound according to any one of clauses 1 to 46.

Clause 53. Method for determining the efficiency of cancer treatment comprising contacting cells with a compound according to any one of clauses 1 to 46.

Clause 54. Method according to clause 52 or 53, wherein the method is performed in vivo.

Clause 55. Method according to clause 52 or 53, wherein the method is performed in vitro.

1. (canceled)

2. A compound of the formula: 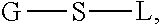
or a stereoisomer, enantiomer, tautomer, prodrug and/or a pharmaceutically acceptable salt thereof,
wherein
G is a glycoside, and a substituted or unsubstituted C₁-C₅ alkyl derivative thereof and/or a substituted or unsubstituted N-acetyl derivative thereof, having a glycosidic bond to S, wherein * represents the binding site between G and S,
S is selected from the group consisting of 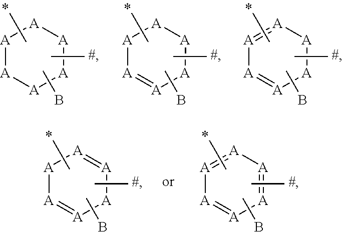
wherein A is independently selected from the group consisting of C, S, N, and O, with the proviso that at least 3 C-atoms are present;
#represents the binding site between S and L;
L is selected from the group consisting of 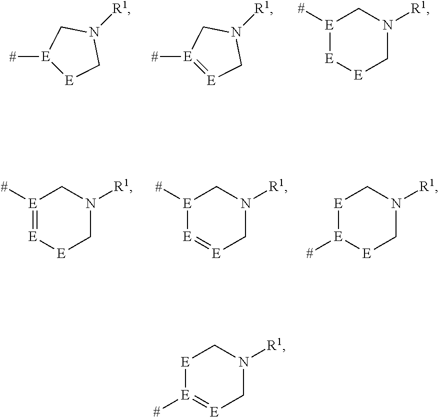
#—CH₂OR², or #—CH₂OOCNHR²;
E is independently selected from the group consisting of N, NH, C, CH, and CH₂;
B and R¹ are independently selected from the group consisting of R³Z, H, F, Cl, Br, I, NO₂, OR⁴, NR⁴R⁵, and substituted or unsubstituted C₁-C₅ alkyl, provided that at least one R³Z is present;
R² is selected from the group consisting of substituted or unsubstituted alkyl, substituted or unsubstituted alkenyl, substituted or unsubstituted aryl, and substituted or unsubstituted heteroaryl; and
R³Z is selected from the group consisting of (CH₂)ₘZ, [(CH₂)ₘO(CH₂)ₙ]ₒZ, [(CH₂)ₘCO(CH₂)ₙ]ₒZ, [(CH₂)ₘOOC(CH₂)ₙ]ₒZ, [(CH₂)ₘNR⁴(CH₂)ₙ]ₒZ, wherein m is 0 to 5, and n and o are independently from each other integers from 1 to 5;
R⁴ and R⁵ are independently selected from the group consisting of H, and substituted or unsubstituted C₁-C₅ alkyl; and
Z is a radioactive detectable label, a radioactive therapeutic residue, a chelator coordinating a radioactive detectable label or a chelator coordinating a radioactive therapeutic residue.

3. The compound according to claim 2, wherein
the substituted or unsubstituted C₁-C₅ alkyl derivative of the glcyoside is a substituted or unsubstituted 5-(C₁-C₅ alkyl)glycoside; and/or
the substituted or unsubstituted N-acetyl derivative of the glycoside is a substituted or unsubstituted 2-(N-acetyl)glycoside, a substituted or unsubstituted 3-(N-acetyl)glycoside, or a substituted or unsubstituted poly(N-acetyl)glycoside; and/or
the glycoside is selected from the group consisting of α-D-glucofuranoside, α-D-mannofuranoside, α-D-fructofuranoside, α-D-glucopyranoside, α-D-mannopyranoside, α-D-galactopyranoside, α-D-fructopyranoside, β-D-glucofuranoside, β-D-mannofuranoside, β-D-fructofuranoside, β-D-glucopyranoside, β-D-mannopyranoside, β-D-galactopyranoside, and β-D-fructopyranoside; and/or
R³Z is selected from the group consisting of Z, [(CH₂)ₘO(CH₂)ₙ]ₒZ, [(CH₂)ₘCO(CH₂)ₙ]ₒZ, NR⁴(CH₂)ₙZ, wherein R⁴ is H, methyl or ethyl, wherein m, n and o are independently from each other integers from 1 to 5; and/or
S is

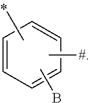

4. The compound according to claim 2, wherein
L is 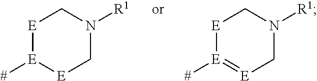
R¹ is independently selected from the group consisting of (CH₂)ₙZ, or O(CH₂)ₙZ, wherein n is 1 to 3;
B is independently selected from the group consisting of H, F, Cl, Br, I, NO₂, OR⁴, NR⁴R⁵, and unsubstituted C₁-C₅ alkyl, provided that at least three H are present, wherein R⁴ and R⁵ are independently selected from the group consisting of H, methyl, or ethyl.

5. The compound according to claim 2, wherein
L is 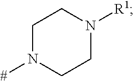
R¹ is independently selected from the group consisting of (CH₂)ₙZ, or O(CH₂)ₙZ, wherein n is 1 to 3, wherein #is in meta-position or para-position to *;
B is independently selected from the group consisting of H, F, Cl, Br, I, NO₂, OR⁴, NR⁴R⁵, and unsubstituted C₁-C₅ alkyl, provided that at least three H are present, wherein R⁴ and R⁵ are independently selected from the group consisting of H, methyl, or ethyl.

6. The compound according to claim 2, wherein
L is #—CH₂OR² or #—CH₂OOCNHR²;
R² is substituted or unsubstituted C₁ to C₈ alkyl, substituted or unsubstituted aryl;
B is independently selected from the group consisting of R³Z, H, F, Cl, Br, I, NO₂, and substituted or unsubstituted C₁-C₅ alkyl, provided that at least one R³Z and at least two H are present are present;
R³Z is selected from the group consisting of —Z, —[(CH₂)ₘO(CH₂)ₙ]ₒZ, —[(CH₂)ₘCO(CH₂)ₙ]ₒZ, —NH(CH₂)ₙZ, wherein m, n and o are independently from each other integers from 1 to 4; and/or
R⁴ and R⁵ are independently selected from the group consisting of H and substituted or unsubstituted C₁ to C₃ alkyl.

7. The compound according claim 2, wherein
L is #—CH₂OR² or #—CH₂OOCNHR², wherein L is in ortho-position or para-position to *;
R² is substituted benzyl;
B is independently selected from the group consisting of R³Z, H, F, Cl, Br, I, NO₂, and substituted or unsubstituted C₁-C₅ alkyl, provided that at least one R³Z and at least two H are present are present;
R³Z is selected from the group consisting of —Z, —[(CH₂)ₘO(CH₂)ₙ]ₒZ, —[(CH₂)ₘCO(CH₂)ₙ]ₒZ, —NH(CH₂)ₙZ, wherein m, n and o are independently from each other integers from 1 to 4, wherein R³Z is in para-position to * or #; and/or
R⁴ and R⁵ are independently selected from the group consisting of H and substituted or unsubstituted C₁ to C₃ alkyl.

8. The compound according to claim 2, wherein the compound is

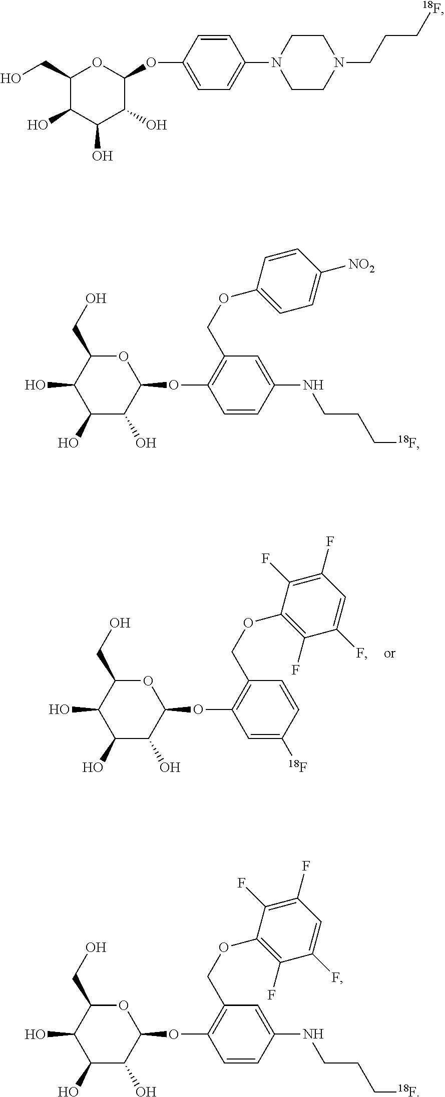

9. The compound according to claim 2 adapted to accumulate intracellularly.

10. The compound according to claim 2, wherein the radioactive detectable label is selected from the group consisting of ¹¹C, ⁴⁰K, ¹³N, ¹⁵O, ¹⁸F, ⁷⁵Br, ⁷⁶Br, ⁸²Rb, ⁶⁸Ga, ⁶⁴Cu, ⁶²Cu, ⁸⁹Zr, ¹²³I, ¹²⁴I, ¹²⁵I, ¹³¹I, ²¹⁰At, ²¹¹At and ¹¹¹In.

11. The compound according to claim 2, wherein the radioactive detectable label is ¹⁸F.

12. The compound according to claim 2, wherein the radioactive therapeutic residue is selected from the group consisting of ³²P, ⁶⁰Co, ⁶⁴Cu, ⁸⁹Sr, ⁹⁰Y ¹⁷⁷Lu, ¹⁸⁶Re and ¹⁵³Sm.

13. A method of using the compound according to claim 2 for surgery comprising administering the compound of claim 2 to a subject undergoing a surgery.

14. A method for detecting cell senescence comprising contacting cells with a compound according to claim 2.

15. The method according to claim 14, wherein the method is performed in vivo.

16. The method according to claim 14, wherein the method is performed in vitro.

17. The method for determining the efficiency of a cancer therapy comprising contacting cells with a compound according to claim 2.

18. The method according to claim 17, wherein the method is performed in vivo.

19. The method according to claim 17, wherein the method is performed in vitro.
